(19) United States
(12) Patent Application Publication
(10) Pub. No.: US 2006/0057636 A1
(43) Pub. Date: Mar. 16, 2006

(54) Composite peptide compounds for diagnosis and treatment of diseases caused by prion proteins

(76) Inventors: Peter Heegaard, Copenhagen (DK); Palle H Jakobsen, Stenlose (DK)

Correspondence Address: EDWARDS & ANGELL, LLP, P.O. BOX 55874, BOSTON, MA, 02205, US

(21) Appl. No.: 10/525,533

(22) PCT Filed: Aug. 25, 2003
(86) PCT No.: PCT/DK03/00555

§ 371 Date: Sep. 14, 2005

(30) Foreign Application Priority Data
Aug. 21, 2002 (DK) PA 2002 01245

(51) Int. Cl. C07K 14/775; G01N 33/53

(52) U.S. Cl. 435/7.1; 530/350; 530/359

(57) ABSTRACT

The present invention relates to diseases caused by prion proteins, Novel composite peptide compounds are disclosed which comprise two or more peptides or peptide fragments optionally linked to a backbone and the peptides or peptide fragments are spatially positioned relative to each other so that they together form a non-linear sequence which mimics the tertiary structure of one or more PrP^{Sc}-specific epitopes as evidenced by the test described herein. The use of such conjugates as immunogens for the production of antibodies that specifically bind to the pathogenic form of a prion protein is revealed. Other uses of the composite peptide compounds are also disclosed, such as use in diagnostic assays, production of antibodies and uses as vaccine immunogens for the prophylactic protection and therapeutic treatment of subjects against transmissible prion disease.

### FIELD OF THE INVENTION

The present invention concerns conjugates (also denoted “composite peptide compounds” in the present context) comprising two or more peptides or peptide fragments optionally linked to a backbone and the peptides or peptide fragments are spatially positioned relative to each other so that they together form a non-linear sequence which mimics the tertiary structure of one or more PrP^{Sc}-specific epitopes as evidenced by the test described herein.

In other words, the conjugates comprise combinations of two or more same or different synthetic peptides or peptide fragments optionally coupled to a backbone such as a backbone peptide, and the two or more peptides or peptide fragments form a non-linear sequence which mimics structural epitopes in the pathogenic form of the prion protein. The invention also relates to the use of such conjugates as immunogens for the production of antibodies that specifically bind to the pathogenic form of the prion protein. In a specific embodiment, the peptides or peptide fragments comprise prion-related peptides bound to a non-dendritic lipopeptide backbone. Preferred uses include use in diagnostic assays of the composite peptide compounds themselves as well as antibodies produced against them and uses as vaccine immunogens for the prophylactic protection and therapeutic treatment of humans against transmissible prion disease.

### BACKGROUND OF THE INVENTION

Prion diseases are peculiar, inevitably deadly neurodegenerative diseases that can occur spontaneously, in inherited forms and by transmission (for a recent review see Prusiner, S. B. Prion Biology and Diseases, Cold Spring Harbor Laboratory Press, New York, 1999). Transmitted prion diseases include Transmissible spongiform encephalopathies (TSE) that are characterised by the occurrence of transmissible protein aggregates (prions) in the brain of the affected individual. The diseases associated with such aggregates encompass sporadic, iatrogenic and familial Creutzfeldt-Jakobs disease (CJD), kuru, Gerstmann-Straussler-Sheinker (GSS) disease, and fatal familial insomnia (FFI) in humans and bovine spongiform encephalopathy (BSE) in cattle, scrapie in sheep, chronic wasting disease (CWD) in deer and elk, and transmissible encephalopathies in mink, cat and other animals.

These deadly diseases all have very long incubation periods (up to several years) and are characterised by the absence of defensive responses (immune responses, inflammatory responses or any other response), except a certain degree of activation of glial cells in the brain quite late in the incubation period.

It is a central dogma of these diseases that they are caused by a misfolded conformer of a normal host protein (the prion proteins, PrP) that when misfolded is also able to transmit disease, i.e. induce the abberant folding of normal prion proteins, eventually leading to disease and pathological changes in the affected individual. Although the participation of the host PrP is central to propagation of disease, prion proteins have a peculiar ability of retaining or transmitting strain characteristics (with specific incubation times, histopathology and glycosylation) even upon the “infection” of a new host. Transspecies infection occurs rarely in nature—with the very notable exception of bovine spongiform encephalopathy, which very probably can be transmitted orally to human subjects and cause new variant CJD—but disease can often be induced by intracerebral injection of pathogenic prion proteins into a new host of a new species; for example scrapie prions (prion from the well known prion disease in sheep) can easily be propagated in hamster brains.

Different prion diseases show different prion deposition patterns in the host; for example, scrapie prions can normally be found in the brain and in the central nervous system in addition to the peripheral nerve system and in immunological organs as e.g. lymph nodes as well as in the blood and in the cerebrospinal fluid. By contrast, BSE prions only occur in the brain and in a few parts of the spinal cord; in the human counterpart of BSE, nvCJD, the pathogenic prion proteins are found in peripheral tissues in addition to the brain.

As no host factors have been described to be 100% specific for infection with pathogenic prion proteins, diagnosis of the disease relies on the detection of the specific, disease-associated proteins found in the prions. As mentioned above, such proteins have been found to be a disease-specific conformer of a normal protein, the prion protein (PrP^{C}). PrP^{C} is a small, membrane-anchored glycoprotein with two N-bound glycans and a C-terminal glycosylphophatidylinositol anchor. It has been found and sequenced in a number of species and the tertiary structure has been determined by NMR- or X-ray spectroscopy for human, murine, hamster and bovine PrP^{C}. PrP^{C} has a very unusual ability to switch between a normal conformation or structure and a pathogenic, disease-associated and transmissible conformation or structure. Such abnormally folded prion proteins are denoted PrP^{Sc}. PrP^{Sc} is remarkable by being extremely stable and very resistant towards proteases, by being able to cause disease and by being able to transmit disease. Proteinase K treatment of PrP^{Sc}, employing conditions which totally degrade most other proteins, including PrP^{C}, leads to a shortened, protease resistant PrP^{Sc} protein molecule weighing 27-30 kD (PrP^{C} has a molecular weight of 33-35 kD). Apart from this and in contrast to PrP^{C}, PrP^{Sc} is insoluble if not treated with harsh chaotropic chemicals and is clearly folded differently from PrP^{C} although the details of the folding of PrP^{Sc} are not yet known.

There are no differences between the amino acid sequences of PrP^{Sc} and PrP^{C}. Thus, prion diseases are linked to the occurrence of pathogenic tertiary structures of otherwise normal host proteins. Transmissible forms of prion diseases demonstrate that such misfolded prion proteins have the ability to transmit disease to a normal host; this is thought to occur by exposure of the normally folded prion protein to the misfolded protein.

BSE emerged in the mid-eighties in Great Britain and quite rapidly evolved into a major epizootic disease, leading to clinical disease in a huge number of cattle (approximately 200,000 until now) with around 20,000 new cases pr. year in the peak years around 1992. The appearance of a new, human prion encephalopathy, nvCJD gave rise to the suspicion that this new human disease was due to exposure to the cattle pathogen and although no definitive proof of this has been obtained, this is now accepted as a distinct possibility. Therefore it is highly desirable to identify and remove animals and meat containing BSE prions from human consumption. To be able to do this, very sensitive, specific and preferably rapid diagnostic assays for detection of PrP^{Sc} in easily obtained samples are needed. Traditional diagnosis is performed by immunohistochemistry on brain sections in combination with histopathology looking for neuropathological changes. These are quite lengthy methods (weeks) and rely on brain tissue from dead animals.

Recently, with the BSE-spurred interest in diagnostic methods for prion protein detection, a number of other methods, based on the use of PrP-specific antibodies in combination with a proteinase K-degradation have evolved, three of those having been evaluated in the EU.

These three EU-validated “rapid” tests (Prionics Western blotting, Enfer (Abbott) ELISA, CEA (BioRad) ELISA) all achieved 100% sensitivity and 100% specificity in the EU-testing (which took place in May 1999). The samples used for this validation were: 336 samples from 300 positive animals (clinical, confirmed cases from UK) and 1064 samples from 1000 negative animals (healthy, adult bovines from New Zealand).

This means that these tests can correctly identify clinically ill animals, i.e. they determine the presence of a disease-specific component in brain (or spinal cord) samples from clinically ill animals. Conversely, healthy animals do not present this component in brain (or spinal cord) samples and hence are not positive in these tests. It is claimed for all three tests (but this was not evaluated in the EU testing) that they can detect the presence of pathogenic prion proteins from around two months before the clinical disease appears.

This, however, is far from ideal, as the real aim of a diagnostic test for a contagious disease is to be able to show the presence of the transmittable agent as soon as it is present in the animal and thus able to transmit to another animal. All three tests fail in this respect as they only show the presence of pathogenic prion protein very late in the infection process, i.e. close to the onset of clinical disease and years after the introduction of the agent into the animal. Ultimatively, the sensitivity should be also high enough for the test to be able to show the presence of infectious prions in asymptomatic carrier animals including other species of animals (pigs, chickens, etc.) which may be able to—but have not been shown to be able to—carry the infection.

### DESCRIPTION OF THE INVENTION

As mentioned above, the known tests suffer from a number of drawbacks. Thus, a major improvement would be to avoid a protease treatment step as this slows down and complicates the assay, decreases the sensitivity of the assay and precludes detection of misfolded and pathogenic, but not protease-resistant forms of PrP; such hypothetical forms may occur during the early phases of infection before enough misfolded PrP has been formed for protease-resistant PrP^{Sc}-aggregates to occur. Furthermore the omission of a protease step would allow immunohistochemical staining with higher sensitivity and higher definition, as tissues would not be affected by the protease.

In one aspect, the present invention provides an improved test for determination of the presence of PrP^{Sc}. The test involves the use of a conjugate comprising two or more peptides or peptide fragments optionally linked to a backbone in such a manner that the fragments are spatially positioned relative to each other so that they form a non-linear sequence which mimics the tertiary structure of one or more PrP^{Sc}-specific epitopes as evidenced by the test described herein.

Given the inadequacies of the current tests there is a need for an improved test, and any improvement is of significant interest with regards to consumer safety. An argument in favour of more sensitive tests is that such tests will allow a much more selective culling and destruction strategy to be employed.

The test should ideally be able to detect “infection” with (or “incubation of”) pathogenic prion proteins in the pre-exponential phase. The concentration of infectious material in this phase is not known and will depend on the source of the sample material, but it is clearly going to be very low and probably absent in some types of samples. Any improvement of sensitivity, is of interest, however, as this will allow the consideration of other types of samples than brain samples.

The possibility of using blood or other types of “non-invasively sampled” material will be a major improvement compared to existing methods and will totally change the surveillance strategies applicable for the control of BSE. As stated above such a method will be dependend on higher assay sensitivity.

There is great concern that BSE hides as an unrecognised infection in sheep or as an infection in sheep classified as scrapie (which is not considered a human pathogen) and there is thus great interest in using a test that will discriminate between BSE and scrapie.

Rapidity is also a major concern, especially for normally slaughtered animals which are to be declared BSE-free. A rapid and sensitive test will pave the way for testing all slaughtered animals, adding to consumer safety and increasing the value of the surveillance. Furthermore, the ideal assay should have a high capacity (high throughput), be easy to use, and be rapid and quantitative. WO93/11155 describes how it is possible to produce antibodies against parts (synthetic or genetically engineered peptides) of the PrP-sequence thereby obtaining PrP-binding molecules that are useful for diagnosis, prophylaxis and treatment of PrP^{Sc}-caused disorders. While the usefulness of coupling one type or several types of such PrP-fragments to carrier molecules for immunization purposes is disclosed, no mention is made of a composite peptide compound containing fragments making up PrP^{Sc}-specific epitopes. Also, the examples disclose antibodies, which do not, generally, distinguish between PrP and PrP^{Sc}. Thus the invention does not in any way teach how to produce PrP^{Sc}-specific antibodies using PrP-fragments.

WO 99/15651 describes an invention that entails the manufacture of PrP^{Sc}-binding substances by using peptide mimotopes based on linear arrangements of one or several peptides being defined by the following substances that were found to display specific binding towards PrP^{Sc}: Monoclonal antibody 15B3, recombinant bovine PrP (rboPrP) and

Congo Red. A set of four peptides defined by peptide mapping of the conformational epitopes corresponding to MAb 15B3 and rboPrP (which bind to the same epitope specific for PrP^{Sc}) is claimed, as is another set of 5 peptides being mapped as Congo Red binders. It is proposed that linear combinations of these peptides will lead to antigens that are specific to PrP^{Sc} and thus will be useful for diagnosis and therapy as well as for detection of substances binding specifically to PrP^{Sc}. However, there is no teaching as to how to achieve an arrangement of these peptide building blocks in a composite epitope, which is PrP^{Sc}-specific.

By contrast, and by utilising a non-conventional way of constructing conjugates, the present invention provides a method for manufacturing conformationally stabilised peptide epitopes. This is done by coupling peptide building blocks in a controlled spacing on a backbone as for example a non-dendritic backbone peptide, whereby the peptide building blocks are brought into close proximity to each other in space, resembling the arrangement of the peptide units found in the PrP^{Sc}-protein and allowing peptide-to-peptide stabilization of the conformation of the individual peptides.

On the basis of WO93/11155 and WO99/15651 it seems that synthetic or recombinant PrP peptide fragments are useful mimics of prion protein specific epitopes and can be used for the production of PrP specific antibodies with the advantage of not having to use potentially dangerous biological material. As summarised above, WO99/15651 discloses the finding that a monoclonal antibody prepared against bovine PrP and able to specifically recognise PrP^{Sc} and not PrP^{c} (also reported by Korth, C., et al., 1997, Nature 390, 74-77) recognises a conformational or non-linear epitope composed of three different segments of the PrP polypeptide chain. The binding of Congo Red, a dye capable of binding specifically to PrP^{Sc} was also mapped to a conformational epitope (five segments), the nature of which was thus also specific for PrP^{Sc}. However, there is no indication that such conformational epitopes specific for the abnormally folded conformer of PrP may be formed by coupling together different peptides deriving from separate parts of the PrP linear sequence, and then be used to produce PrP^{Sc}-specific antibodies. No specific methods are presented, on how to prepare such substances and no example demonstrating the production of such antibodies by this method is described. Also, no instructions are given as to the best mode of preparing such composite or conformational epitopes by linearly combining different PrP peptide molecules.

As the accurate three-dimensional structure of PrP^{Sc} is not known at present, it is not possible to produce structural analogues by conventional methods and conventional methods for producing and displaying peptides as those described in WO 98/37210 and WO 93/15651 do not allow such structural epitopes to be mimicked by peptides. The shortcomings of these methods are due to the fact that only linear arrangements of two or more different peptides are described, precluding the desired structural mimicry of the conformational epitopes that are specific for PrP^{Sc} and which are constituted by a composite of a number of peptide stretches from different parts of the polypeptide chain.

The inventors have found from inspecting current models for the abnormal fold of the PrP^{Sc} conformer (Korth, C., et al., 1997, Nature 390, 74-77, & FIGS. 4-6) that it is simply not possible to mimic the conformational epitopes specific for PrP^{Sc} by just combining the constituent peptides linearly; this is especially due to the fact that, in order for such an epitope mimic to be completely specific for PrP^{Sc}, which is an especially preferred object of the invention, the complete conformational epitope has to be represented by the peptide mimic. In the models available for PrP^{Sc}, specific, conformational epitopes (FIGS. 4-6) such epitopes always comprises peptide fragments that 1) have a stabilised secondary structure (beta-strand like), 2) runs in different directions of each other and 3) are to some degree positioned side-by-side rather than end-to-end (Korth, C., et al., 1997, Nature 390, 74-77, Wille, H., et al., 2002, Proc. Natl. Acad. Sci., USA 99, 3563-3568). Except from 1) none of these features are obtainable by the linear peptide constructs of WO99/15651 and WO 93/11155.

By contrast, the present invention provides a method for the controlled spatial organisation of two or more different PrP-related peptides in an immunogenic and conformationally stabilised peptide construct characterised by being non-linear, and by containing PrP peptides in a predetermined, non-linear arrangement mimicking the spatial positions of the corresponding sequences in the PrP protein fold. It is especially constructs which mimic the spatial positions of PrP peptides in the abnormally folded PrP^{Sc} conformer which are the object of the invention; exemplary considerations on the design of such constructs are given in Example 3.

In a preferred embodiment the peptide construct further comprises a lipidic moiety conveying the construct with a balanced amphipathicity that allows the construct to be soluble in a benign aqueous buffer and at the same time to be surface active and able to form stable aggregates in such solvents. It is particularly preferred that this amphipaticity makes the construct immunogenic without the addition of powerful adjuvants as e.g. Freund's as demonstrated in WO97/38011 with a wide number of peptide antigens. In this instance, antibodies can be produced by simply inoculating the animal with a efficient amount of peptide construct (as for example 10 μg to 200 μg) in a sufficient number of times (e.g. 3 or 4 times) by an adequate route, preferably subcutaneous, intravenous, intraperitoneal or intramuscularly. This is preferable to the use of highly surface active adjuvants as there is no risk of perturbing the conformational structure of the peptides in the construct.

Riley et al., 2002, Protein Engineering 15(6), 529-537 describes a linear recombinant dimeric construct of human PrP further containing a FLAG peptide fusion segment, and also describes the efficient expression of the corresponding glycosylated PrP-dimer in a yeast expression system. This is an example of a linear combination of two full-length PrP molecules which is furthermore shown not to resemble PrP^{Sc}.

In WO 97/10505 the desired antibodies reactive against PrP^{Sc} are obtained by preparing phage libraries expressing antibody fragments binding PrP^{Sc}, obtaining genes expressing PrP^{Sc}-reactive antibodies from PrP^{Sc} immunised PrP^{0/0} (PrP-knock-out) mice. While this document describes in great detail the need for PrP^{Sc} specific antibodies in order to develop better diagnostic methods it only discloses using the whole protein for this procedure and thus does not anticipate the present invention which is directed towards using smaller fragments of the prion protein.

### DEFINITIONS

In the present context the term “prion” means a proteinaceous infectious particle, i.e. the infectious agent of prion diseases.

The term “PrP” denotes the prion protein, without specification of its conformation or aggregation state.

The term “PrP^{C}” denotes the prion protein, a normal host protein having a molecular weight 33-35 kDa, fully digestible by proteinase K.

The term “PrP^{Sc}” denotes the abnormally folded and pathogenic form of the prion protein, being trimmed to a 27-30 kDa fragment by proteinase K-treatment.

The term “backbone” denotes a carrier molecule, preferably a peptide and preferably of a low molecular weight, i.e. preferably below 3 kDa, said molecule comprising at least one attachment point for the coupling of PrP peptide fragments. As it appears later, backbones may be conformationally stabilised, nondendritic backbones as described in WO 97/38011.

The term “non-linear” used in connection with a conjugate of the present invention describes a peptide chain which does not consist of a single one-dimensional string of amino acids which runs from N→C without branching, but rather a number of chains of amino acids which are connected together at branching points to give a non-linear structure. In other words non-linear conjugates are characterised by having more than one direction of the peptide chain. Non-amino acid portions may be included at branching points or between amino acid residues.

The term “lipophilic moiety” when used in connection with the present invention denotes a branched or unbranched, saturated or unsaturated, substituted or unsubstituted chain of from 5 to 30 carbon atoms.

The term “non-dendritic peptide backbone” denotes a synthetic peptide having a defined number of derivatization points in defined positions along its peptide chains, and the derivatization points are functional groups of specific amino acid residues in the peptide chain.

The term “bovine PrP” denotes a peptide identified as follows (SEQ ID No:1) (whole sequence, including signal peptide (underlined); numbering is according to this sequence throughout the text) or polymorphs thereof:

MVKSHIGSWI LVLFVAMWSD VGLCKKRPKP GGGWNTGGSR
YPGQGSPGGN RYPPQGGGGW GQPHGGGWGQ PHGGGWGQPH
GGGWGQPHGG GWGQPHGGGG WGQGGTHGQW NKPSKPKTNM
KHVAGAAAAG AVVGGLGGYM LGSAMSRPLI HFGSDYEDRY
YRENMHRYPN QVYYRPVDQY SNQNNFVHDC VNITVKEHTV
TTTTKGENFT ETDIKMMERV VEQMCITQYQ RESQAYYQRG
ASVILFSSPP VILLISFLIF LIVG

Analogously, SEQ ID No: 2 relates to Ovine PrP and SEQ ID No:3 to Human PrP. The terms also include any polymorphs thereof.

### ABBREVIATIONS

BMPS: N-[β-maleimidopropyloxy]succinimide ester
bo: bovine
BSA: bovine serum albumin
BSE: bovine spongiform encephalopathy
CJD: Creutzfeldt-Jakob disease
CWD: chronic wasting disease
ELISA: enzyme-linked immunosorbent assay
ESI: Electrospray ionization
FFI: fatal familial insomnia
Fmoc: fluorenylmethoxycarbonyl
GSS: Gerstman-Straussler-Scheinker disease
HOBt: hydroxybenzotriazole
HPLC: high pressure liquid chromatography
MS: mass spectrometry
Mtt: methyltrityl
NMM: N-methylmorpholine
NMP: N-methyl pyrrolidone
Npys: 3-nitro-2-pyridinesulfenyl
nvCJD: new variant CJD
OD: optical density
OPD: ortho phenylene diamine
Pbf: 2,2,4,6,7-pentamethyl dihydrobenzofuransulfonyl
PBS: phosphate-buffered saline
PrP: prion protein (no specific conformer implied)
SPDP: 3-(2-pyridyldithio)propionic acid hydroxysuccinimide ester
TBS: Tris-buffered saline
TBTU: 2-(1-H-benzotriazole-1-yl)-1,1,3,3-tetramethyluronium tetrafluoroborate
TFA: trifluoroacetic acid
TNBS: trinitrobenzenesulfonic acid
TSE: transmissible spongiform encephalopathy

Peptides or Peptide Fragments

In one aspect, the invention relates to a conjugate comprising two or more peptides or peptide fragments optionally linked to a backbone and the peptides or peptide fragments are spatially positioned relative to each other so that they together form a non-linear sequence which mimics the tertiary structure of one or more PrP^{Sc}-specific epitopes as evidenced by the following test.

The test is made in order to secure that the conjugate has the desired structure to enable formation of antibodies specifically directed to PrP^{Sc} without recognizing PrP^{c}. The test involves the use of two samples (a positive and a control). The first sample (positive sample) is from an animal being a carrier of PrP^{Sc} and the second sample (control) is from an animal of the same species as the first animal being a carrier of PrP^{c} but a non-carrier of PrP^{Sc} (healthy non-carrier). The test comprises detection of any PrP^{Sc} in the samples by i) contacting the samples with an antibody obtained by immunizing an animal with the conjugate, ii) measuring any PrP^{Sc} that is bound to the antibody, the test being carried out without any use of Proteinase K.

Such a test may specifically be performed as described in the following example which is meant to illustrate the principles of testing, and a person skilled in the art will know how to adjust test conditions for the testing of other samples:

A 10% homogenate is prepared from a brain sample from a healthy cow and from a cow with BSE, respectively, by homogenizing with PBS with 2% sarcosyl. The two samples are tested for the presence of protease-resistant PrP^{Sc} by analysis in the standard Prionics Western blotting assay to confirm the presence of PrP^{Sc} in the BSE-sample (sample B) and the absence of PrP^{Sc} in the normal sample (sample A).

Samples are then analysed by ELIFA, enzyme-linked immuno assay, as described by Korth et al. (1999, Meth. Enzymol. 309, 106-122). This method employs filtration of samples through a blotting membrane, followed by detection of PrP^{Sc} on the membrane by the antibodies being tested. Here, homogenates (untreated or treated with proteinase K (20 μg/ml, 30 minutes, 37 degrees Celcius)) diluted with PBS (in the, range of 50-500 times) are filtered through the membrane by vacuum suction. The membrane is then blocked with 2% Tween 20 in TBS for 5 minutes and subsequently incubated with the antibodies that are being tested, typically titrated from 100 μg/ml and downwards, using a volume of 50 μl and an incubation time of 2 hours at room temperature. After washing the membrane is developed by alkaline phosphatase- or peroxidase conjugated second antibody against mouse (or rabbit as adequate) immunoglobulins (1 hour incubation at room temperature), followed by visualization, preferably by a chemiluminescent substrate (ECL, Amersharh).

This is done with and without proteinase K-treatment. The antibodies of the present invention will be able to react specifically with PrP^{Sc} with no reaction with PrP^{C} and the reaction with PrP^{Sc} will be substantially identical with and without proteinase treatment, although it is to be expected that some protease-sensitive PrP^{Sc} will also be detected, increasing the signal of the untreated sample in comparison with the treated sample.

Another useful test method uses bead-coupled antibody to catch PrP directly from a brain homogenate and then test the protease-resistance of the material being caught, e.g. by the standard Prionics Western blotting assay for detection of protease resistant PrP^{Sc}. It is expected that the antibodies of the invention will exclusively or almost exclusively catch protease resistant PrP^{Sc} and no PrP^{C} from a sample B brain homogenate while no PrP^{C} will be caught from the sample A normal brain homogenate. In the sample B homogenate it is to be expected that the antibodies of the invention will also bind soluble and less protease resistant forms of PrP^{Sc} not being detected by standard assays relying on proteinase resistance. This assay can be performed by coupling a purified version of the antibodies of the invention to activated Dynabeads, for example Dynabeads M-280 (tosyl-activated). This is done in 0.1 M borate pH 9.5 using 0.1 mg/ml of the antibody, 100 μl antibody for 1 ml Dynabead suspension. This is then incubated with tilt rotation for 24 hours at 37 degrees C. followed by washings in PBS with 0.1% BSA and blocking in 0.2 MTris, pH 8.5 with 0.1% BSA for 4 hours under the same conditions. The antibody-coated beads are then washed in PBS/BSA and brought into contact with the homogenate samples, either treated with proteinase K as above or untreated and incubated by tilt rotation for 2 hours at room temperature, using 100 μl bead suspension for 100 μl brain homogenate sample. After washing, beads are suspended in sample buffer and subjected to Western blotting, using a standard antibody to develop the blot (Prionics 6H4 for example) and visualization by chemiluminescent assay. All washing operations are performed using a magnetic device from Dynal for separating beads from solvent. Blood samples can be tested in substantially the same way. Such samples can be whole blood, plasma, serum, extracted buffy coat, extracted red blood cells or any other kind of treated or untreated blood sample.

In a second embodiment, the invention relates to a conjugate comprising two or more peptides or peptide fragments optionally linked to a backbone and the peptides or peptide fragments are spatially positioned relative to each other so that they together form a non-linear sequence which mimics the tertiary structure of PrP^{Sc} and has the same or a higher degree of conformational sensitivity to PrP^{Sc} as one or more conformationally sensitive regions of PrP^{C} as evidenced by the test described herein.

More specifically, in a conjugate according to the invention the peptides or peptide fragments comprise from 2 to 150 amino acids such as from 4 to 100, from 4 to 75, from 5 to 75, from 4 to 60, from 6 to 60, from 7 to 50, from 4 to 50, from 4 to 40, from 8 to 40, from 4 to 30, from 9 to 30, from 4 to 20 or from 10 to 20 amino acids.

In a conjugate according to a specific embodiment of the invention, at least one of the two or more peptides or peptide fragments is a prion peptide or a prion peptide fragment.

Furthermore, as it appears from the examples herein, in another embodiment of a conjugate according to the invention, all peptides or peptide fragments are prion peptides or prion peptide fragments.

The prion peptides or prion peptide fragments of the present invention may have a primary structure corresponding to PrP of a mouse, a rat, a pig, a human, a sheep, a cow, a hamster, a mule deer, a white tailed deer or a Rocky Mountain elk or polymorphs or fragments thereof. Additionally, the prion peptide or prion peptide fragment may have a primary structure corresponding to a bovine PrP SEQ. ID No. 1, a ovine PrP SEQ. ID No. 2, a human PrP SEQ. ID No. 3, or polymorphs or fragments thereof.

The peptides coupled to the backbone are preferably coupled covalently in a controlled way by methods known to a person skilled in the art of peptide chemistry and as detailed below and in the examples and may comprise one or several of the following PrP-derived peptides, and peptide fragments and mixtures thereof:

(numbering is for bovine PrP; it is to be understood that any of the given bovine PrP-peptides can be substituted by the equivalent peptide from PrP of another species, especially human and ovine)

| | |
|---|---|
| bo88-104: HGG GWGQPHGGGG WGQG | SEQ ID NO:4 |
| bo94-105: QPHGGGG WGQGG | SEQ ID NO:5 |
| bo100-111: G WGQGGTHGQW N | SEQ ID NO:6 |
| bo101-115: WGQGGTHGQW NKPSK | SEQ ID NO:7 |
| bo103-121: QGGTHGQW NKPSKPKTNM K | SEQ ID NO:8 |
| bo134-151: GGLGGYM LGSAMSRPLI H | SEQ ID NO:9 |
| bo173-186: YYRPVDQY SNQNNF | SEQ ID NO:10 |
| bo217-229: MERVVEQMCITQY | SEQ ID NO:11 |
| bo229-247: YQ RESQAYYQRG ASVILFS | SEQ ID NO:12 |
| bo231-242: RESQAYYQRG AS | SEQ ID NO:13 |

Especially interesting are peptides and peptide fragments from the following regions of PrP as they constitute regions that are selectively exposed in PrP^{Sc} and not in PrP^{C}:

| | | |
|---|---|---|
| bo114-123: | GGTHGQW NKPSKPKTNM KHV | SEQ ID NO:14 |
| bo153-171: | GSDYEDRY YRENMHRYPN Q | SEQ ID NO:15 |
| bo139-176: | YM LGSAMSRPLI HFGSDYEDRY | SEQ ID NO:16 |
| | YRENMHRYPN QVYYRP | |

In another embodiment, the above three peptides are synthesized on β-strand inducing building blocks known in the art and available commercially, including dibenzofuran turn mimics, before coupling to the backbone molecule. One example of such as construct is: SAMSRPLIHFG-dib-SDYEDRYYR, in which “dib” represents a 4(2-aminoethyl) 6-dibenzofuranpropionic acid residue, and the amino acids representing region 143-162 (SEQ ID NO:17) in bo PrP.

Other examples of this kind of peptides include:

| | | |
|---|---|---|
| bo143-162: | SAMSRPLIHFGSDYEDRYYR | SEQ ID. 17 |
| bo114-123: | GGTHGQW NKPSKPKTNM KHV | SEQ ID NO:18 |
| bo153-171: | GSDYEDRYYRENMHRYPN Q | SEQ ID NO:19 |
| bo139-176: | YM LGSAMSRPLI HFGSDYEDRY | SEQ ID NO:20 |
| | YRENMHRYPN QVYYRP | |

all of which may be synthesized with a β-strand inducer in the N-terminal end.

In one particular embodiment the following three 15B3 binding peptides are coupled together, one copy of each peptide on the backbone in a controlled orientation ensuring their side-by-side positioning:

| | | |
|---|---|---|
| bo153-159: | GSDYEDR | SEQ ID NO:21 |
| bo173-181: | YYRPVDQYS | SEQ ID NO:22 |
| bo226-237: | ITQYQRESQAYY | SEQ ID NO:23 |

Another embodiment uses the following peptides in a similar arrangement:

SEQ ID NO:24 bo41-44: YPGQ and peptides from PrP comprising this fragment.

SEQ ID NO:25 bo octarepeat: GWGQPHGGGWGQPHGG, and peptides being shorter parts of this sequence.

Also of interest is the use of the following peptides or shorter parts thereof in a composite peptide compound:

(bo102-152 + 199-242)

SEQ ID NO 45

GQGGTHGQW NKPSKPKTNM KHVAGAAAAG AWGGLGGYM LGSAMSR
PLI HF DC VNITVKEHTV TTTTKGENFT ETDIKMMERV VEQMCI
TQYQ RESQAYYQRG AS
(bo146-152 + 199-236)

SEQ ID NO 46

SRPLI HF DC VNITVKEHTV TTTTKGENFT ETDIKMMERV VEQMC
ITQYQ RESQAY
(bo146-152 + 199-232)

SEQ ID NO 47

SRPLI HF DC VNITVKEHTV TTTTKGENFT ETDIKMMERV VEQMC
ITQYQ RE
(bo146-152 + 199-230)

SEQ ID NO 48

SRPLI HF DC VNITVKEHTV TTTTKGENFT ETDIKMMERV VEQMC
ITQYQ
(bo146-152 + 199-228)

SEQ ID NO 49

SRPLI HF DC VNITVKEHTV TTTTKGENFT ETDIKMMERV
VEQMCITQ
(bo146-152 + 199-227)

SEQ ID NO 50

SRPLI HF DC VNITVKEHTV TTTTKGENFT ETDIKMMERV
VEQMCIT

Specifically for use in a “peptide-based assay” the following peptides and peptide fragments are used together with structure relaxing substances (as e.g. the [RG₂]n peptide (n=2-4), or Kₙ (n=3-6)) in an optimal solvent ensuring the highest possible conformational sensitivity of the peptide to contact with PrP^{Sc} as described in detail below:

SEQ ID NO:26 Peptides derived from bo90-145: G GWGQPHGGGG WGQGGTHGQW NKPSKPKTNM KHVAGAAAAG AVVGGLGGYM LGSAM

Additional peptides that are useful for inclusion in a composite peptide compound include:

| | | |
|---|---|---|
| bo102-113: | GQGGTHGQWNKP | SEQ ID NO:27 |
| bo108-119: | GQWNKPSKPKTN | SEQ ID NO:28 |
| bo134-151: | GGLGGYMLGSAMSRPLIH | SEQ ID NO:29 |
| bo136-154: | LGGYMLGSAMSRPLIHFGS | SEQ ID NO:30 |
| bo231-242: | RESQAYYQRGAS | SEQ ID NO:31 |
| bo97-112: | GGGGWGQGGTHGQWNK | SEQ ID NO:32 |
| bo153-171: | GSDYEDRYYRENMHRYPNQ | SEQ ID NO:33 |
| bo151-160 | CGSDYEDRY | SEQ ID NO:34 |
| bo173-181: | YYRPVDQYS | SEQ ID NO:35 |
| bo227-237: | TQYQRESQAYY | SEQ ID NO:36 |
| bo238-254: | QRGASVILFSSPPVILL | SEQ ID NO:37 |
| bo173-186: | YYRPVDQYSNQNNF | SEQ ID NO:38 |
| bo226-237: | ITQYQRESQAYY | SEQ ID NO:39 |
| bo204-215: | TKGENFTETDIK | SEQ ID NO:40 |
| bo153-160: | GSDYEDRY | SEQ ID NO:41 |
| bo155-173: | DYEDRYYRE | SEQ ID NO:42 |
| bo173-181: | YYRPVDQYS | SEQ ID NO:43 |
| bo227-240: | TQYQRESQAYYQRG | SEQ ID NO:44 |

Also interesting peptides are peptides being parts or fragments of the above sequences. A β-strand inducing building block may be introduced in the amino acid sequence of the above-mentioned peptides or peptide fragments. In a specific embodiment, at least two of the two or more peptides or peptide fragments have identical amino acid sequences. In another preferred embodiment a T-helper epitope peptide is included in the construct to secure efficient T-cell help upon immunization with the peptide. Therefore, the present invention concerns a conjugate wherein at least one of the two or more peptides or peptide fragments is a T-cell helper epitope.

As described above the defining difference between the normal prion protein, PrP^{C} and the abnormal, pathogenic prion protein, PrP^{Sc} at the molecular level is a difference in the folding at the secondary and tertiary structure levels of the PrP protein whereas there are no differences in the amino acid sequence. This results in a protein with pronounced differences in physico-chemical features especially big increases in insolubility and protease resistance and also biological effects as neurotoxicity and—given the right circumstances—the ability to bind to other PrP^{C} protein molecules and causing them to misfold. No structure has been determined experimentally for the PrP^{Sc} form, but it is however known that the conformational changes enclose an increase in β-structure on the expense of α-structure as well as tertiary structure changes thought to occur mainly at the N-terminal part of the protease resistant part of PrP^{Sc}. However, until now only one such PrP^{Sc}-specific conformational epitope (15B3) has been identified and mapped to three different parts of the polypeptide chain that are brought into close proximity of each other in PrP^{Sc} in contrast to the case in PrP^{C} where these parts of the polypeptide chain are widely separated. Also an assembled epitope binding to the amyloid-specific dye Congo Red has been mapped to five parts of the PrP polypeptide chain.

The inventors expect that many more PrP^{Sc}-specific epitopes exist, all of them, probably conformational (non-linear), as any linear epitope will also exist in PrP^{C}.

In an embodiment of the present invention, the conjugate is non-linear and has the formula:
(F)ₘ(X)ₚ
wherein

F is independently the same or different prion peptide or prion peptide fragment;

X is the same or different amino acid residue or peptide;

m is an integer from 2 to 10 inclusive;

and p is an integer from 0 to 10 inclusive;

such that X and F together form a conjugate, provided that the resulting conjugate is not prion peptide or a prion peptide fragment.

Furthermore, in those cases where p is different from zero, the conjugate may be a chain of F moieties substituted or interrupted by X moieties. Alternatively, the conjugate may be a chain of X moieties substituted or interrupted by F moieties. In a particular case, the conjugate has the structure
F₁—F₂—F₃—F₄—Fₘ
wherein

F and m are as defined above;

such that 2-10 F moieties are linked to each other, provided that the resulting conjugate is not prion peptide or a prion peptide fragment

The above structure is intended only to mean those conjugates which fulfil the requirements for non-linearity, as described herein. Therefore, F moieties may be linked through their side chains, for instance.

The present invention also concerns a conjugate which is non-linear as described herein which has the structure: 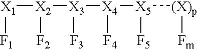
wherein

F₁, F₂, F₃, F₄, F₅ . . . (F)ₘ are each independently the same or different prion peptides or prion peptide fragments;

X₁, X₂, X₃, X₄, X₅ . . . Xₘ are the same or different amino acid residues or peptides each linked to an F moiety and each being attached by peptidic bonds to the preceding and the following X;

m is an integer from 2 to 10 inclusive;

and p is an integer from 0 to 10 inclusive;

provided that the resulting conjugate is not prion peptide or a prion peptide fragment.

In the above structure, the C-terminus of a first F may be connected to the C-terminus of a second F optionally via a backbone or an amino acid side chain of the first and/or the second F. Alternatively, the N-terminus of a first F may be connected to the N-terminus of a second F optionally via a backbone or an amino acid side chain of the first and/or the second F

Furthermore, a first F may be connected to a second F via a side-chain residue of an amino acid in the second F, or through amino acid side chains in the X moieties. In a particular embodiment of the above structures, m is 3. Additionally, one or more F in the above structure may be substituted with a lipophilic moiety Lip.

Backbone

It is an object of the invention to provide such composite peptide compounds in a form where they are conformationally well-defined i.e. conformationally stabilised and at the same time soluble and preferably immunogenic by administration with adjuvants known to a person skilled in the art of animal and human vaccination. In the invention described herein this may be achieved by coupling minimal peptides that constitute a PrP^{Sc} specific conformational epitope to a non-dendritic peptide backbone (see FIG. 1). The resulting derivatised non-dendritic peptide backbone is soluble and conformationally stable and the compound is well suited for immunization. Hence, in a specific embodiment, a backbone is contained in the conjugate. The linking makes it possible to ensure that the peptide or peptide fragments are spatially positioned relative to each other so that they together mimic the tertiary structure of one or more PrP^{Sc}-specific epitopes.

The conjugates of the present invention may comprise a peptide backbone whereon peptides corresponding to PrP-segments are coupled to form an epitope mimicking an epitope found in PrP^{Sc}. The backbone may be a non-dendritic peptide backbone of the type disclosed in WO9738011 having two or more attachment points onto which the peptides are coupled. In a further embodiment at least three such as 4, 5, 6, 7, 8, 9 or 10 peptides or peptide fragments are linked to a backbone.

The backbone according to the present invention comprises more than one accessible functional group and is furthermore derivatized in one or more locations by a lipophilic moiety. In other words, the backbone is a lipopeptide. It is possible to describe such backbones with the structure
(Lip)ₓ-(A)ₙ
wherein

A is an amino acid which may be the same or different and may contain one or more attachment points;

n is an integer from 2 to 150 such that (A), is a chain of amino acids which may be branched or linear;

Lip is a lipophilic moiety which is linked to A through a bond or a linker;

and x is an integer from 1 to 10 such that 1-10, same or different, Lip could be joined to the backbone.

In further embodiments of the above structure, n may be an integer from 2 to 150, such as e.g. from 2 to 130, from 2 to 100, from 2 to 80, from 2 to 60, from 2 to 50, from 5 to 50, from 5 to 40, from 10 to 40, from 15 to 40, from 20 to 35.

In another embodiment, the backbone has the structure
Lip-A₁-A₂-A₃-A₄-A₅-(A)ₙ
wherein

A₁, A₂, A₃, A₄, A₅ . . . (A)ₙ are each independently the same or different amino acids, each of which may contain one or more attachment points;

Lip is a lipophilic moiety which is linked to A₁ through a bond or a linker

n is an integer from 2 to 150.

Again, n may be an integer from 2 to 150, such as e.g. from 2 to 130, from 2 to 100, from 2 to 80, from 2 to 60, from 2 to 50, from 5 to 50, from 5 to 40, from 10 to 40, from 15 to 40, from 20 to 35.

In embodiments of the present invention, A₁ is the N-terminus or the C-terminus of the backbone.

As mentioned previously, the term “lipophilic moiety” when used in connection with the present invention denotes a branched or unbranched, saturated or unsaturated, substituted or unsubstituted chain of from 5 to 30 carbon atoms. More suitable chains are 7-17 carbon atoms long. In an embodiment particularly preferred for immunization purposes, the backbone peptide furthermore comprises a fatty acid, such as a derivative of a naturally occurring or synthetic fatty acid, such as propinic acid, butyric acid, valeric acid, capric acid, heptanic acid, caprylic acid, nonanic acid, capryl, undecanic acid, lauric acid, tridecanic acid, myristic acid, myristoleic acid, myristelaidic acid, pentadecanic acid, palmitic acid, phytanic acid, palmitoleic acid, palmitelaidic acid, heptadecanic acid, stearic acid, petroselinic acid, oleic acid, elaidic acid, ricinoleic acid, ricinelaidic acid, vaccenic acid, trans-vaccenic acid, linoleic acid, conjugated linoleic acids, linoelaidic acid, linolenic acid, γ-linolenic acid, conjugated linolenic acids, nonadecanic acid, arachidic acid, eicosenic acid, homo-γ-linolenic acid, arachidonic acid, heneicosanic acid, behenic acid, erucic acid, tricosanic acid, lignoceric acid, nervonic acid and cerotic acid. The fatty acids may be introduced as lipoamino acids (Toth, I, WO 94/02506; Olive, C., et al., 2003, Inf. Imm. 71, 2373-2383). An example of a lipid moiety is the immunostimulating tripalmitate moiety (Wiesmüller et al., 1992, Int.J. Pept. Prot. Res. 40, 255-260). In a particularly interesting embodiment of the present invention, Lip is a palmitate (palm) residue.

In the above definitions; the linker joining Lip to A may be selected from the group consisting of ether, thioether, amine, ester, amide, carbamate, thioamide, thiocarbamate, urea and thioester.

In one embodiment of the present invention at least one A as defined above is Lys. Alternatively, at least one A is Cys.

An attachment point denotes the functional groups in the backbone peptide available for derivatisation. The backbone may contain two or more attachment points for linking of two or more peptides or peptide fragments. The backbone may also contain one or more Lys and/or Cys residue for linking of peptides or peptide fragments to the backbone (see generalised structures in FIG. 1).

The backbones of the invention are generally peptides comprising a number of attachment points of which some subsets may be protected orthogonally to others and further comprising a lipophilic moiety in one end of the molecule. In one embodiment backbone peptides are 5-50 amino acids in length and comprise 2-10 attachment points. Particularly preferred are backbone peptides between 10 and 25 amino acids in length and with 2-5 attachment points. A preferred embodiment of such backbone peptides comprises peptides with different protecting groups having different chemical stabilities in the attachment points, allowing the selective deprotection and the chemical reaction of one subset of attachment points with a PrP-derived peptide before the deprotection and derivatisation of the other attachment point subset, allowing the controlled incorporation of two different PrP peptides as described in example 5 (see also FIGS. 1B and 7). In addition to two such subsets of attachment points being orthogonally protected, the functional groups of the subsets can also be different, allowing the coupling of two different peptides in opposite directions. In one instance, the attachment point on the backbone comprises amino groups. In a variation of this structure, the backbone contains carboxylates as attachment point. In yet another variation, thiols are included as the attachment points. In still another variation carbonyls, haloacetyls, hydrazides, aoxoacyl, amino oxyacetyl (i.e. hydroxylamine), cysteine, maleimide-groups are derived by chemical methods from primary amino groups.

The conjugates according to the invention comprise a backbone onto which the prion-specific peptides are attached. Such backbones may be branched lipopeptides with molecular weights of at the most about 10,000 kD such as at the most about 5,000 kD or at the most about 3,000 kD or at the most about 1,000 kD and are characterized by being soluble in benign buffers (i.e. aqueous medium), by displaying a well-defined (i.e. stable) conformation under physiological conditions, and by being immune stimulating. The backbone may be branched or non-branched. The backbone peptide part can contain a number of different attachment points for coupling prion peptides and they can be designed with different protecting groups with different and complementary chemical stabilities to allow the selective deprotection of a selected number of attachment point at selected positions in the backbone. This allows the coupling of specific peptides at specific positions to be performed. The lipophilic group of the backbone according to the present invention may be connected to the N-terminus or the C-terminus of the backbone.

Chemical methods for derivatisation of peptides that have been shown to be especially useful for the coupling of peptides to backbone are haloacetylation, especially bromoacetylation of aminogroups leading to a compound that is highly reactive with thiol groups resulting in a stable thioether, maleimide-derivatisation of amino groups eg. by maleimide succinimide heterobifunctional compounds (of which a range is available from Pierce), leading to thiol reactive compounds also forming stable thioethers and, finally dipyridyldisulfide derivatisation of thiol groups leading to a mixed disulfide that is highly reactive with a thiol group, leading to a new mixed disulfide (linking together backbone and peptide). Examples of maleimide and activated disulfide methods are described in Example 2.

Non-dendritic backbones suitable for the present use are such backbones as disclosed in WO 97/38011. Such backbones are suitable for use for immunization purposes. They are synthesized by solid phase peptide chemical synthesis and can be used for coupling of other substance while deprotected or selectively deprotected and still attached to the solid phase.

Examples of suitable non-dendritic backbone peptides include:

a) palm-KVAKLEAKVAKLEAKVAKLEAKG

b) palm-VACLEAKVACLEAKVACLEAKGKGKG

c) palm-VAKLEAKVACLEAKVACKGKG

d) palm-VAKLEAKVACLEAKVAKLEAKVAC

in which K may be selectively side-chain deprotectable compared to the other protected amino acid residues present in the peptide; and

KRGGKRGGK-(palm)

palm-VAKLEAKVACLEAKVACKG K G

palm-VAKLEAKVACLEAKVAKLEAKVACKG KG

in which K may be selectively side-chain deprotectable compared to the other protected amino acid residues present in the peptide; and

palm-PrP

palm-PrP fragment

GSDYEDRYYK-(palm)

YMLGSAMSRPK-(palm)

In all of the above examples, the peptide side chains at one or more positions may be optionally protected by protecting groups, and K may be selectively side-chain deprotected compared to the other protected amino acid residues present in the peptide. The fragment “-(palm)” describes a “palm” moiety which is attached to either the C-terminal amino acid or the N-terminal amino acid of the above fragments, possibly through a side chain in said amino acid.

A particular preferred embodiment of the invention features the use of a first PrP-derived peptide as the backbone carrying a second and different PrP-derived peptide coupled onto the first peptide as described in example 5. An interesting way to combine two such different peptides in a two-peptide construct is to perform the coupling to allow the two peptides to adopt opposite directions in the construct thereby mimicking the positions and directions of the peptide segments in the structure of the PrP^{Sc} specific epitope being mimicked (see example 5 and FIG. 6).

Equally preferred is another group of backbone peptides being themselves prion protein-derived and being furthermore coupled to a lipophilic moiety in one end and being able to react chemically with another PrP-peptide, preferably in a position at the other end of the molecule compared to the position of the lipophilic group. In an embodiment of the present invention, one or more prion peptides or prion peptide fragments are coupled via their N-terminal amino acids to one of the backbones described above. In two particular embodiments, the backbone is GSDYEDRYYK-(palm) or YMLGSAMSRPK-(palm)

It certain cases, the epitopes of the present invention can bind to 15B3, Congo Red, PrP and/or PrP peptides. This type of recognition is described in U.S. Pat. No. 5750362.

It is also an object of the present invention to provide conjugates comprising non-dendritic backbone peptides, which are not lipidated.

Additional examples of such conjugates are compounds in which the backbone peptide is itself a prion peptide carrying another prion peptide. A specific example of this is:

GSDYEDRYYK-(palm) coupled to CYYRPVDQYSN

YMLGSAMSRPK-(palm) coupled to CGSDYEDRYYRE

These constructs are preferably coupled together through the cysteine thiol group to a thiol-reactive heterobifunctional coupling reagent (for example BMPS or SPDP) coupled to the N-terminal amino group of the other peptide.

A further object on the present invention is to provide conjugates comprising prion peptides coupled to a backbone, said prion peptides being characterised by having included in their sequence an accessory residue stabilising the secondary structure of said backbone. An example of such an accessory molecule is 4(2-aminoethyl) 6-dibenzofuranpropionic acid (a β-strand inducer).

A non-dendritic peptide backbone can be synthesized by standard Fmoc-chemistry using active esters or in situ/preactivated amino acids (e.g. TBTU/HOBt/NMM in NMP) amino acid active esters or symmetric anhydrides (see e.g. Atherton, E., and Sheppard, R. C., 1989, “Solid phase peptide synthesis. A practical approach”, IRL Press) on a suitable solid phase which include commercially available polymers as Rink amide resin, Rink-amide PEGA resin, Chlorotrityl resin, Wang resin, NovaSyn resin as well as any other polymers that can be derivatized and are insoluble in the solvents used for synthesis. Depending on the type of solid-phase polymer, the substitution should be between 0.01 and 0.1, preferably 0.05 to 0.1 and even more preferably about 0.05 mequivalents pr. gram solid phase; Novasyn is typically used at about 0.05 mequivalents pr. gram. The commercially available Novasyn KB resin, which contains the HMB-linker (4-hydroxy-methyl benzoic acid), hydroxyl groups at around 0.150 mequivalents pr. gram are available for peptide coupling. A substitution of 0.05 mequivalents pr. gram is then achieved by performing the esterification of the C-terminal amino acid residue of the backbone with a short incubation time, resulting in partial substitution only of the hydroxyl groups of the linker.

Subsequently, and before alpha-amino deprotection of the coupled amino acid, additional free hydroxyl groups are blocked by incubation with a large surplus of acetic anhydride in the presence of the esterification catalyst N,N dimethylaminopyridine (DMAP). After backbone synthesis, HMB can be cleaved to form the free acid, the carboxamide, the acylhydrazine, etc. The linkage of the backbone to the solid-phase can be, but is not limited to be, constituted by base-cleavable linkers. Any other scheme employing orthogonal or graded protection of side-chains, protection of α-aminogroups and stability of the linker is amenable. In a preferable method lysine residues are orthogonally protected in their epsilon-amino groups as e.g. Fmoc-K(Mtt)OH, FmocK(Dde)-OH and Fmoc-K(Aloc)-OH, in which side chain protecting groups are removable by 1% TFA, 2% hydrazine and Pd(0)(catalytic hydrogenation), respectively. After synthesis, alphaamino lipid-coupling and selective lysine-side chain demasking, synthesis or coupling of branch peptides or moieties can then be accomplished with no risk of interference from unmasked functional groups in the side chains of other amino acids in the backbone.

As stated previously, examples of suitable non-dendritic backbone peptides include:

| | |
|---|---|
| a) | palm-KVAKLEAKVAKLEAKVAKLEAKG |
| b) | palm-VACLEAKVACLEAKVACLEAKGKGKG |
| c) | palm-VAKLEAKVACLEAKVACKGKG |
| d) | palm-VAKLEAKVACLEAKVAKLEAKVAC |

As an example of how the composite peptide compounds of the invention can be produced using the non-dendritic backbone peptide of the above types, the nondendritic backbone peptide a) can be selectively deprotected on K, then derivatised with N-[β-maleimidopropyloxy]succinimide ester (BMPS), and subsequently reacted with a mixture of cysteine-containing synthetic prion peptides, leading to a composite peptide compound consisting of the non-dendritic backbone peptide molecule onto which a stoechiometric mixture of prionpeptides are coupled. In another example, employing structure b) this method is reversed, using synthetic BMPS-derivatised prion-peptides that are coupled to the deprotected cysteine side-chain thiol groups of the non-dendritic backbone peptide. In both cases a composite peptide compound containing an equal mixture of (bo145-151), (bo165-173) and (bo219-229) is preferred.

In c) and d) K is selectively deprotected and the ε-amino group used as an anchoring point for the stepwise chemical synthesis of one prion peptide, using a standard protocol with the nondendritic backbone peptide still attached to its solid phase. Hereafter the whole peptide is deprotected and used for coupling of synthetic BMPS-derivatised prion peptides. In this way the exact positioning of the individual peptides are fully controlled. A particularly preferred embodiment of such composite peptide compounds is a nondendritic backbone peptide c) onto which is coupled (bo145-151) on the selectively deprotected lysine ε-amino group followed by BMPS-coupled (bo165-173) and BMPS-coupled (bo219-229) on the two cysteine thiol groups (mixture). A further preferred embodiment comprises the nondendritic backbone peptide d) carrying bo(145-151) on the ε-amino groups of the two selectively deprotected lysine residues and BMPS-coupled bo(165-173) bound through the cysteinyl thiol groups to the nondendritic backbone peptide.

### OTHER ASPECTS OF THE INVENTION

In one particular preferred embodiment of the invention the composite peptide compound assumes a PrP^{Sc} specific conformation only upon the exposure to PrP^{Sc} or PrP^{Sc} aggregates. This conformation is then detected by specific antibodies or by change in fluorescence accomplished by incorporating fluorescent molecules in the composite peptide compound. Hence, a conjugate according to the present invention may further comprise a marker. The marker may be a fluorescent molecule, biotin, avidin, streptavidin, chemiluminescent molecule and the like.

This allows the direct use of the composite peptide compound of the invention for detection of PrP^{Sc}. A test can be performed to ensure that the composite peptide compound has the desired structure in order to be able to undergo a conformational change when contacted with PrP^{Sc}, but not when contacted with PrP^{C}. The test involves the use of two samples (a positive and a control). The first sample (positive sample) contains PrP^{Sc} and the second sample (control) contains PrP^{C}. The test comprises detection of PrP^{Sc} by means of a conformational change of the conjugate by i) incubation of the conjugate in a structure-relaxing solvent with the two samples, respectively, ii) measuring any conformational change of the conjugate by conformation-specific antibodies or by detection of changes in the fluorescence of an environmentally sensitive fluorophore coupled to the conjugate.

The feasibility of using the composite peptide compound of the present invention as direct probes for the presence of PrP^{Sc} as opposed to PrP^{C} is based on the fact that prion-derived peptides can induce PrP^{C} to become PrP^{Sc} in vitro under conditions of large surplus of peptide and provided the peptide is partly unfolded (Prusiner, S., et al., 1995, U.S. Pat. No. 5,750,361); this, in turn, means that the unfolded peptide participates directly in this transformation, initiating it by binding to PrP^{C}. Based on this observation, it is an object of the present invention to provide PrP^{C} peptide-containing composite peptide compounds which mimic parts of PrP^{C} that, upon contact with PrP^{Sc} under appropriate solvent conditions (structure relaxing solvent conditions) undergo a conformational change that can be observed through fluorescent labels on the peptide. The basic idea is to provide conformationally facilitated PrP^{C}-peptides and allowing them to interact with PrP^{Sc} in a conformationally relaxing solvent. “Conformationally facilitated” means that the PrP^{C} peptides of the compound are arranged in order to have an enhanced tendency to form a β-sheet structure, but being in a random coil in the non-provoked compound. Thus, a peptide from the conformationally sensitive part of PrP (for bo PrP this will be the 90-145 region) is brought into contact with a sample in a structure relaxing solvent, or employing other forms of structure relaxing treatments (one preferred example being sonication as reported by Saborio et al. (Saborio, G. P., 2001, Nature 411, 810-813)). If the sample contains PrP^{Sc} this will influence the conformation of the composite peptide compound and this conformational change can, in turn, be detected by the conformation-specific antibodies of the present invention or through changes in the fluorescence of built-in and environmentally sensitive fluorophor-quench pairs. The effect can be enhanced by inserting structure-relaxing peptide sequences into the prion peptides; such peptide sequences include [RG₂]ₙ, where n=2-4, as well as Kₙ, where n=3-6, and they are preferably placed in one end of the prion peptide and preferably in the end being attached to the backbone peptide. The final design of a peptide compound of this type will be characterised by a subtle balance between structure stabilization and relaxation to allow the conformational shift occurring upon exposure to PrP^{Sc} to become as big as possible. Also the influence of PrP^{C} in the sample will have to be taken into consideration, and conditions will have to be employed which minimize binding of the peptide to this form of the prion protein.

It is furthermore expected that an assay of this type can be rendered species specific by choosing species specific sequences from the relevant region of the prion protein. This is possible because it is to be expected that the conformational sensitivity of a given prion peptide sequence built into a composite peptide compound of the present invention will be higher towards PrP^{Sc} from the species in question or, if present in another species, higher towards the specific PrP^{Sc} type (“strain”) formed in response to the original PrP^{Sc} in this particular species. This principle of “strain persistency” has been observed and reported in the scientific literature in a number of cases (for example various scrapie PrP^{Sc} in hamsters, see e.g. Safar, J., et al., 2000, Arch. Virol. Suppl. 16, 227-235) and the principle and its implications for the composite peptide compound assay is outlined in FIG. 8.

Also, the composite peptide compound can be used as such, after appropriate labelling for the detection of PrP^{Sc} as it was found that a certain class of claimed compounds of this type bound specifically to this form of the molecule, changing its own conformation upon the binding of PrP^{Sc}

It is an object of the present invention to provide PrP^{Sc} mimics that can be used to produce PrP^{Sc}-specific antibodies in laboratory animals, said antibodies intended for use in diagnostic assays for prion diseases, i.e. for sensitive detection of PrP^{Sc}. The present invention is therefore directed towards a method for identifying PrP^{Sc} by means of a substance which undergoes conformational change when contacted with PrP^{Sc}, the method comprising

i) incubation of the substance with PrP^{Sc} in a structure-relaxing solvent,

ii) measuring any conformational change of the substance by conformation-specific antibodies or by detection of changes in the fluorescence of an environmentally sensitive fluorophore coupled to the substance.

Another intended use is as vaccine immunogen for the vaccination of subjects and animals against transmissible prion diseases. As detailed above, there is a very small risk of inducing antibodies against the normal prion protein of the host as the composite peptide compounds of the invention are designed to represent conformational epitopes of PrP^{Sc} only. Therefore, conjugates according to the present invention may be used in the production of antibodies. Such antibodies may be specific for PrP^{Sc}.

The conjugates of the present invention are PrP^{Sc}-mimics that can be easily produced chemically in large scale, and are non-infectious and easy to handle by anyone skilled in the art of peptide chemistry and immunology. The conjugates of the present invention may therefore readily be used in medicine, such as in vaccines. Preferred uses include directly as reporter substances in PrP^{Sc}-assays, utilizing either antibody detection or a flurophor signal generation detection system. Also preferred is the use of such compounds for the production of diagnostic antibodies in laboratory rodents, and the use of said antibodies to specifically detect PrP^{Sc} in a high background of PrP^{C}. A highly preferred use of assays based on such antibodies is for the detection of minute amounts of PrP^{Sc} in ante-mortem types of samples including blood, saliva, urine and the like. Another preferred use of such compounds is for production of vaccines as well as therapy against transmissible prion diseases targeting specifically the PrP^{Sc} conformer of the prion protein. Apart from these medical uses it is clear that the PrP^{Sc}-specific antibodies and composite peptide compounds of the present invention will also be highly useful for research purposes, one particular preferred use being the high-throughput screening of pharmaceuticals interfering with PrP^{Sc} formation or directly destroying PrP^{Sc} complexes.

It is envisaged that the surprising finding of the ability of a normal animal to mount an antibody response against the PrP^{Sc}-mimicking composite peptide compounds of the present invention are due to the lack of presence of such PrP^{Sc}-specific conformational epitopes in such animals. Thus, the encounter of such an animal with the conformationally stabilised PrP^{Sc}-mimicking composite peptide compound of the present invention leads to a host reaction directed against the “foreign”, intruding structure. By contrast the conventional dogma is that it is very hard to make normal animals produce antibodies against PrP, including PrP^{Sc} as this is not seen as “foreign” by the animal, and thus it is normally considered necessary to use PrP^{0/0} animals for this purpose (see for example WO97/10505). By contrast, the immunization of normal animals with the structure-stabilised composite peptide compounds of the present invention takes advantage of the exact fact that PrP is not “foreign” to a normal animal and therefore such an animal will produce antibodies solely against structures that are not found in normal PrP which is exactly what the composite peptide compound represent. It is furthermore envisaged that the inability of a normal animal to produce antibodies against PrP^{Sc} particles is due to the insolubility of natural PrP^{Sc} making its removal from the body a job of other parts of the immune system not including antibodies. In contrast, the composite peptide compounds of the present invention in addition to being conformationally stable are carefully designed to be fully soluble in aqueous, physiological buffers and are therefore fully able to induce antibody production in the immunized host.

Surprisingly, the conjugates of the present invention can be used for immunization of animals in which the PrP gene has not been removed or modified, resulting in highly reactive and specific antibodies that can react with the pathogenic conformers of PrP, i.e. PrP^{Sc}. The composite peptide compounds of this invention are constructed in such a manner that the different peptides that are included in the composite peptide compound collectively represent a conformational epitope corresponding to a conformational (non-linear) epitope only found in the abnormally folded form of PrP.

It is also the object of the present invention to provide antigens that mimic parts of the abnormally folded prion protein, PrP^{Sc}, that are not found in the normal form of the prion protein, PrP^{C}. The invention also encompasses the use of such antigens for the production of antibodies that are specific for PrP^{Sc}, by immunization with said antigens and diagnostic assays employing such antigens and antibodies.

The present invention is directed towards a method for the production of antibodies against PrP^{Sc}, the method comprising immunizing an animal with a conjugate as described previously. The animal may be selected from the group consisting of mice, rats, rabbits and poultry, and the antibodies may be monoclonal or polyclonal antibodies. The invention is also directed towards antibodies, which, when contacted with a mixture of PrP^{Sc} and PrP^{C}, interact with PrP^{Sc} without substantially interacting with PrP^{C}.

The invention also describes a method for the manufacture of such antigens in which a conjugate having a polypeptide chain is produced. The polypeptide chain corresponds to an assembled epitope at the surface of the PrP^{Sc} and the epitope is not present in PrP^{C}. The conjugate is generally produced by chemical peptide synthesis by chemically combining a number of linear peptides, each corresponding to a linear stretch of amino acids in the prion protein into a composite molecule. The linear peptide building blocks can be produced by chemical peptide synthesis or optionally by expression by a recombinant expression system.

The conjugates of the invention can be tested to secure that the conjugates have the desired structure to enable formation of antibodies specifically directed to PrP^{Sc} without recognizing PrP^{C}. The test involves the use of two samples (a positive and a control). The first sample (positive sample) is from an animal being a carrier of PrP^{Sc} and the second sample (control) is from an animal of the same species as the first animal being a carrier of PrP^{C} but a non-carrier of PrP^{Sc} (healthy non-carrier animal). The test comprises detection of any PrP^{Sc} in the samples by i) contacting the samples with an antibody obtained by immunizing an animal with the conjugate, ii) measuring any PrP^{Sc} that is bound to the antibody, the test being carried out without any use of Proteinase K. Optionally, the sample may be treated with Proteinase K before it is contacted with antibody.

The origins of suitable samples for this test are animals such as a cow, a sheep, a monkey, a human, a pig, poultry, a mouse, a rat, a hamster, a mule deer, a white tailed deer or a Rocky Mountain elk or other mammal. The sample may be extracted from the brain, muscles, lymphoid tissues, spinal cord, bone marrow, nerve tissue and blood cells, and the animal may be alive or dead.

Also, by providing a sensitive means of detection of malfolded prion proteins without using protease resistance as a parameter, the present invention allows the detection of PrP^{Sc} in biological material where it is present in trace amounts only, e.g. in blood, plasma, serum, urine, lymph, cerebrospinal fluid, saliva and tear fluid etc.

An important feature of the invention is the possibility of achieving a satisfactory degree of species specificity by using the exact amino acid sequence of the species in question. It is also possible, however; to construct conjugates that are not specific for one species but will correspond to prion proteins from a number of species by constructing the peptides using consensus or homologous amino acid stretches of the prion protein, or by providing a mixture of molecules each representing a different form of the prion protein.

Furthermore, the conjugates provided herein can be used for the screening of various substances, including combinatorial chemical libraries, for PrP^{Sc}-binders. This can be achieved by assays employing blocking of PrP^{Sc}-specific antibodies or blocking of PrP^{Sc}-sensitive composite peptide compounds. Substances defined by such methods are useful leads for the generation of drugs that block the change of PrP^{C} into of PrP^{Sc}.

The present invention is also directed towards pharmaceutical composition or a vaccine composition comprising a conjugate or an antibody as described herein. The conjugates or antibodies of the present invention may be used for treating and/or preventing Creutzfeldt-Jakobs disease, kuru, Gerstmann-Straussler-Sheinker disease, fatal familial insomnia and transmissible spongiform encephalopathies, such as bovine spongiform encephalopathy in cattle, scrapie in sheep, chronic wasting disease in deer and elk and transmissible encephalopathies in mink, cat and other animals.

Still further aspects of the invention appear from the appended examples and claims.

### LEGENDS TO FIGURES

FIG. 1

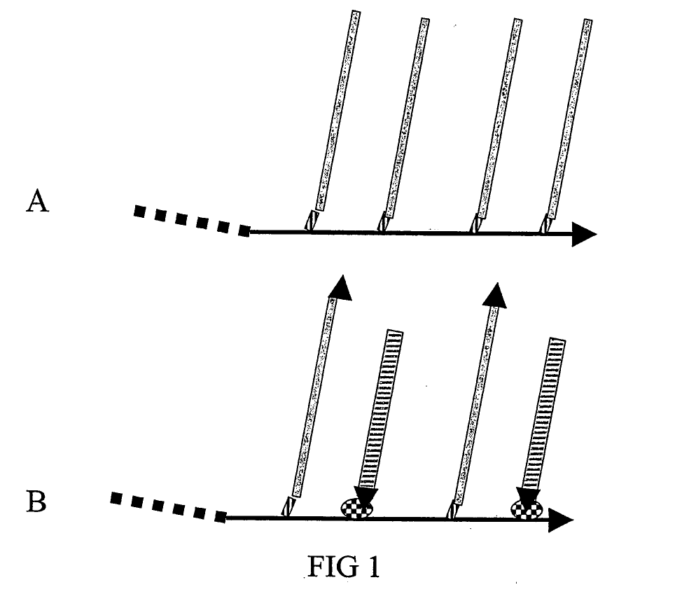

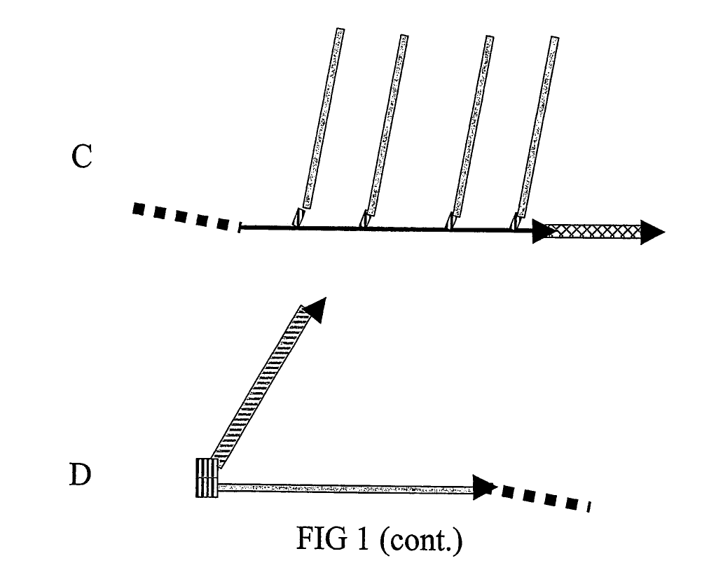

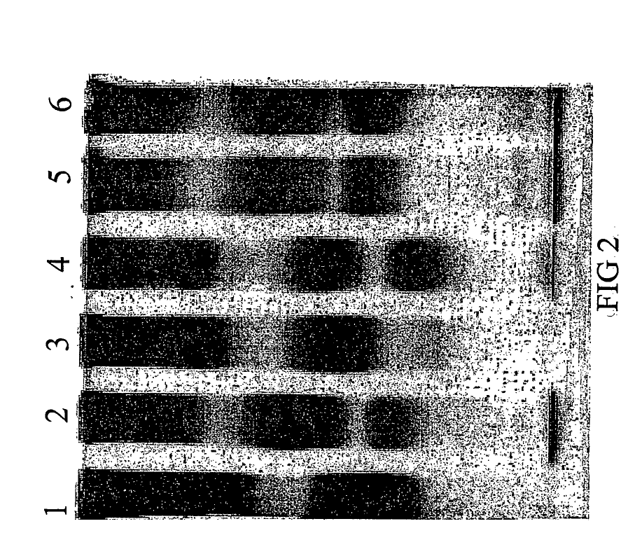

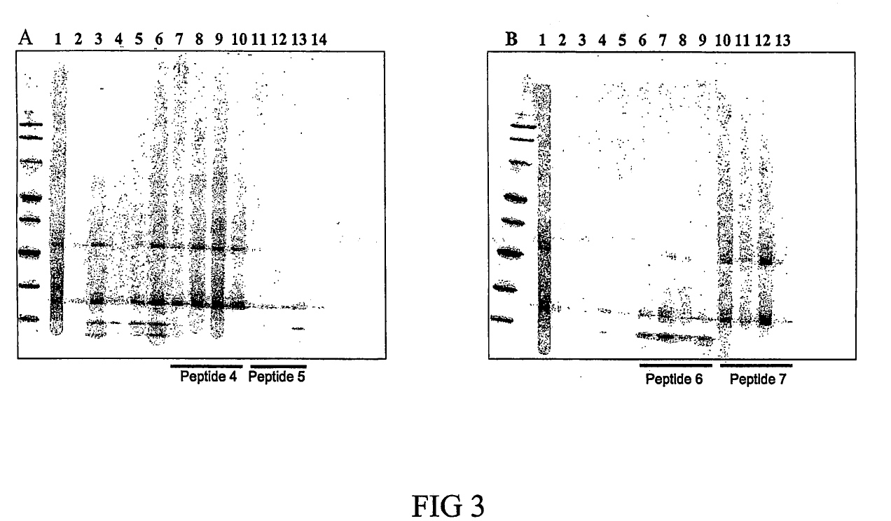

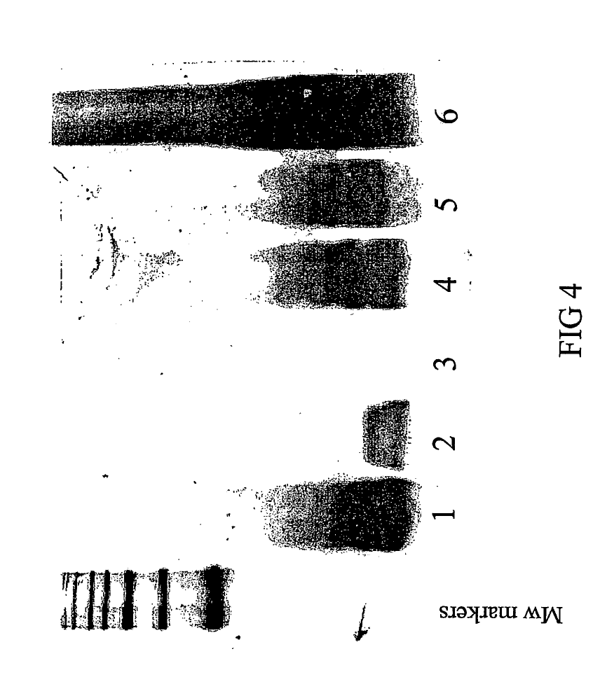

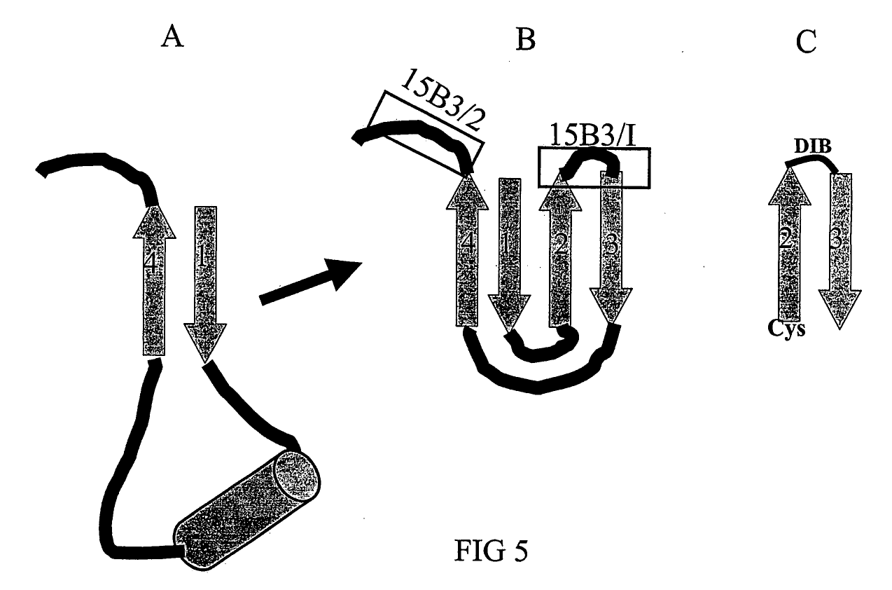

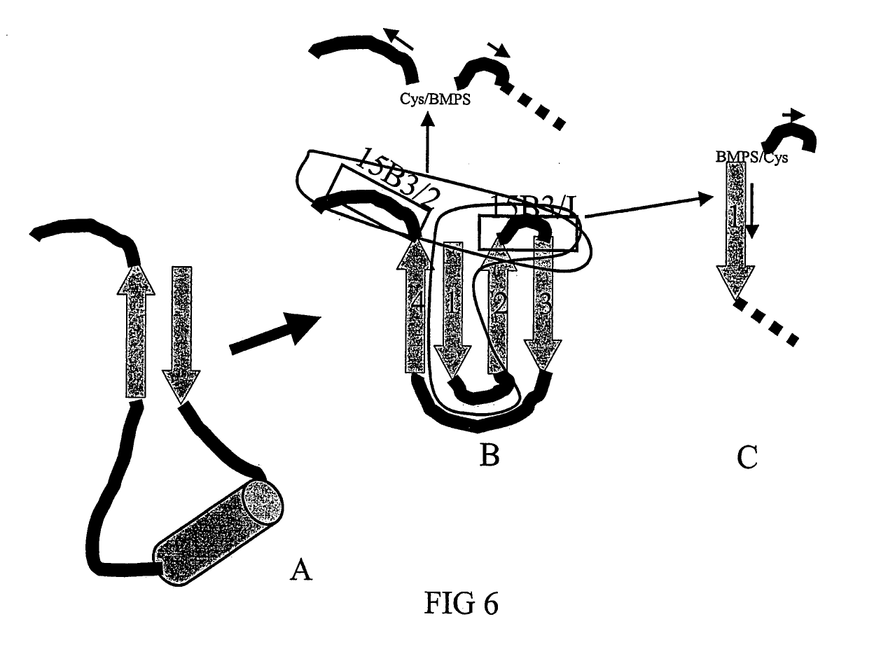

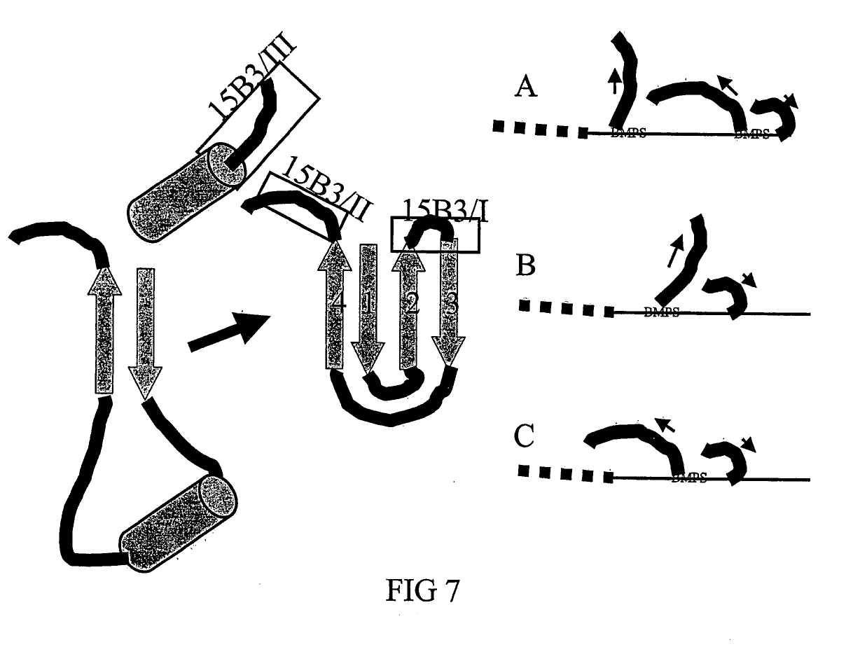

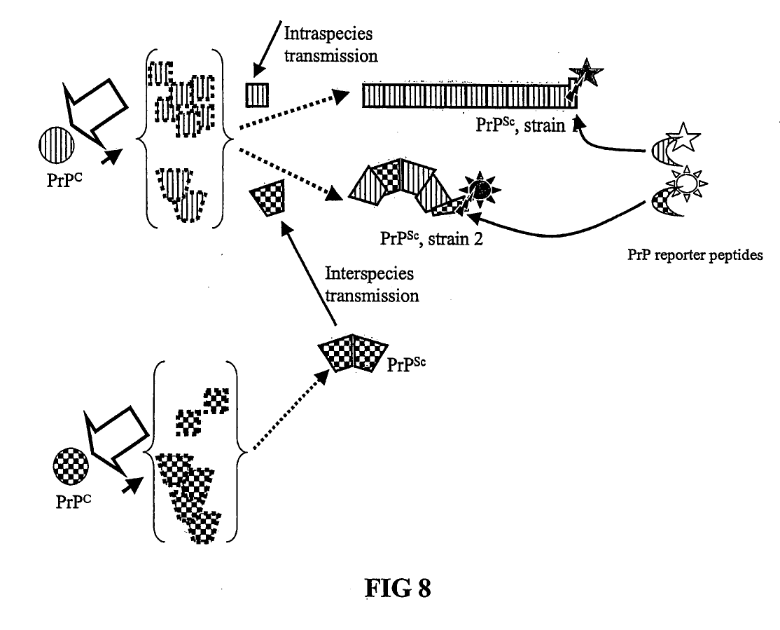

A: General conjugate structure showing backbone peptide (solid horizontal arrow), attachment points (small,striped rectangles), hydrophobic moiety (fatty acid)(dotted line) and coupled antigenic PrP-peptides (grey, elongated rectangles).

B: Conjugate structure with backbone with two different types of attachment points, each coupling a different PrP-peptide. In this case the two different peptides are also coupled in opposite directions.

C: Conjugate structure with a backbone onto which PrP-peptides are coupled and which comprises an auxiliary segment, e.g. an efficient Th-epitope peptide (cross-hatched rectangle).

D: An equally preferred conjugate structure in which the backbone is itself a PrP-peptide, carrying another PrP-peptide; arrangement of backbone, the other PrP-peptide and fatty acid is shown.

Arrowheads indicate directions of peptide chains (N to C). If no arrowhead is present the direction can be either way

FIG. 2

Peptide-ovalbumin conjugates analysed by SDS-PAGE and silver staining. The conjugates were obtained by the method described in Example 1, i.e. using maleimide derivatised ovalbumin and coupling with cysteine-containing peptides. Lane 1: maleimide-activated ovalbumin (control); lanes 2-6: Each lane shows the result of coupling a different peptide. It is obvious from this analysis that the ovalbumin used for coupling (Sigma, grade V, A-5503) contains several molecular species of very different molecular weights. It is also seen that the modification with the peptide affects all ovalbumin-related bands, but to a different degree depending on the peptide. For some of the peptides a small molecular weight product occurs, presumably peptide-peptide polymers.

FIG. 3

Western blotting to test antibodies obtained by immunization with peptide-ovalbumin conjugates for reactivity with recombinant bovine PrP (Prionics). Recombinant (E. coli) bovine PrP, 10 μg/ml, was run in a non-reducing SDS-PAGE system (NuPAGE, 4-12%, MOPS, Novex) and blotted onto an Immobilon (Millipore) membrane.which was then incubated with the various mouse sera (end bleeds) at 1/200 unless otherwise specified. The following sera were analysed

| | | | |
|---|---|---|---|
| A: | 1: 6H4 1/5000 (Prionics) | B: | 1: 6H4 1/5000 (Prionics) |
| | 2: 0-bleed | | 2: Buffer |
| | 3: X-peptide, end-bleed | | 3: 0-bleed |
| | 4: 6H4-peptide, 0-bleed | | 4: 0-bleed |
| | 5: 6H4-peptide, end bleed | | 5: Pep5, mouse 4 |
| | 6: Pep1, mouse 3 (NB: 1/100) | | 6-9: Pep6, mouse 1-4 |
| | 7-10: Pep4, mouse 1-4 | | 10-13: Pep7, mouse 1-4 |
| | 11-13: Pep5, mouse 1-3 | | |
| | 14: Buffer | | |

(see example 1 for sequences of the peptides used).

(6H4 is a monoclonal antibody against PrP from Prionics (positive control))

FIG. 4

Peptide-backbone model constructs analysed by Western blotting with anti-peptide antibody detection on Western blot analysis (nonreduced 20% polacrylamide gel) of a conjugate consisting of the PrP peptide acetyl-CWGQGGTHGQWNKPSK coupled to the backbone CVAKLEAKVACLEAKVAKLEAKG (with and without N-terminal palmitate). The coupling was performed with dipyridyidisulfide as described in Example 2. The blot was visualised using a mouse antiserum raised against the same peptide coupled to ovalbumin (see Example 1). Lanes contain: 1: Peptide alone, 2: activated peptide alone, 3: backbone alone, 4: backbone coupled with peptide, 5: palmitoylated backbone alone, 6: palmitoylated backbone coupled with peptide.

FIG. 5

The N-terminal part of the globular domain of PrP^{C} is schematically depicted to the left, indicating the presence of helix 1 and the two-beta strand sheet as experimentally determined to constitute the structure of PrP^{C} in this region. After transformation into PrP^{Sc} the resulting putative structure shown to the right of the arrow occurs, transforming the original helix 1 into two additional beta strands teaming up with the original two beta-strands (1 and 4) to form a four beta-strand sheet as indicated. Two parts of the 15B3 epitope (specific for PrP^{Sc}) are marked by the boxes.

To the right in the figure is indicated a PrP^{Sc}-specific peptide mimic, employing a beta-strand promoting turn-residue (DIB, see text). This peptide specifically mimics PrP^{Sc}, as this structure is obviously not found in PrP^{C}.

FIG. 6

Left-hand side of the figure is as in FIG. 5. In the middle part is indicated the positions of two peptide mimics and the positions of two 15B3-epitopes. The two peptide mimics are produced by coupling two different peptides from different parts of the chain together in opposite directions, thereby specifically mimicking PrP^{Sc}-specific structures (peptide chain directions are indicated by the small arrows, fat dotted line symbolizes palmitate), see B (top) and C.

FIG. 7

Left-hand side of the figure is as in FIG. 5. The middle part indicates the position of all PrP^{Sc}-specific 15B3-epitopes and also shows the position of helix 3 and its C-terminal extension in the PrP^{Sc} protein. To the right are indicated (from the top) positions and directions of prion peptides in a composite peptide compound carrying three different prion peptides, and two examples of composite peptide compounds carrying two different prion peptides. In these structures the peptide representing 15B3/1 is always present as this is the PrP^{Sc}-defining part of the epitope, resulting in PrP^{Sc}-specific composite peptide compounds when combined with any other parts of the 15B3-epitope or both parts at the same time.

FIG. 8

Schematic figure depicting the relationship of prion proteins from one species (all vertically hatched objects) to prion proteins from another species (all chequered objects), the circular figure depicting PrP^{C}, i.e. the normal conformer and pathogenic conformers (PrP^{Sc}) being depicted by various quadrangles.

It is hypothesized that PrP^{C} exists in a very skewed equilibrium with various unstable PrP^{Sc} conformers (the PrP^{Sc} conformer population), one specific PrP^{Sc} conformer, however, being the preferred one in each species (the PrP^{Sc} strain characteristic for that species).

Furthermore, the figure shows how PrP^{Sc} from one species can “infect” another species leading to stabilization of unstable PrP^{Sc} into pathogenic, stable PrP^{Sc} aggregates containing PrP^{Sc} conformers of the inoculum type (“strain”). Thus, a specific PrP^{Sc} conformer preferentially binds to and stabilises the corresponding specific PrP^{Sc} conformer of the other species even if this conformer is only a minor part of the PrP^{Sc} conformer population of that species. Note that the development of PrP^{Sc} conformers of this type is slower than the development of PrP^{Sc} conformers of the type typical for the species being infected.

Also shown is how a labelled, conformationally sensitive peptide (“reporter peptide”) can be used to detect PrP^{Sc} in a sample by specific binding to that PrP^{Sc} leading to a measurable change in the conformationally sensitivite label of the peptide.

As the preference for forming a particular PrP^{Sc} conformer is thought to be governed by the exact amino acid sequence of the prion protein of the species in question, a peptide built from that particular sequence will also show preference towards being bound by that particular conformer even if they are built from another amino acid sequence, i.e. from the prion protein of another species. Thus the reporter peptide will always indicate the “strain-type” of the infecting PrP^{Sc} to the extend that this type is replicated in the host species PrP^{Sc}.

Methods

Test—Immunization

As it appears from the appended claims, a conjugate according to the invention comprises two or more peptides or peptide fragments optionally linked to a backbone and the peptides or peptide fragments are spatially positioned relative to each other so that they together form a non-linear sequence which mimics the tertiary structure of one or more PrP^{Sc}-specific epitopes. A suitable test for demonstrating that the structure is suitable is as follows:

The test is made in order to secure that the conjugate has the desired structure to enable formation of antibodies specifically directed to PrP^{Sc} without substantially recognizing PrP^{C}. The test involves the use of two samples (a positive and a control). The first sample (positive sample) is from an animal being a carrier of PrP^{Sc} and the second sample (control) is from an animal of the same species as the first animal being a carrier of PrP^{C} but a non-carrier of PrP^{Sc}.

The test comprises detection of any PrP^{Sc} in the samples by i) contacting the samples with an antibody obtained by immunizing an animal with the conjugate, ii) measuring any PrP^{Sc} that is bound to the antibody, the test being carried out without any use of Proteinase K.

Test—Peptide Assay

In another embodiment the invention relates to a conjugate comprising two or more peptides or peptide fragments optionally linked to a backbone and the peptides or peptide fragments are spatially positioned relative to each other so that they together form a non-linear sequence which mimics the tertiary structure of PrP^{Sc} and has the same or a higher degree of conformational sensitivity to PrP^{Sc} as one or more conformationally sensitive regions of PrP. A suitable test to demonstrate the correct structure is given in the following:

The test is made to secure that the conjugate has the desired structure in order to be able to perform a conformational change when contacted with PrP^{Sc}, but not or at least not to the same extent when contacted with PrP^{C}. The test involves the use of two samples (a positive and a control). The first sample (positive sample) contains PrP^{Sc} and the second sample (control) contains PrP^{C}.

The test comprises detection of any conformational change of the conjugate by i) incubation of the conjugate in a structure-relaxing solvent with the two samples, respectively, ii) measuring any conformational change of the conjugate by conformation-specific antibodies or by detection of changes in the fluorescence of an environmentally sensitive fluorophore coupled to the conjugate.

### EXAMPLES

The following examples demonstrate the feasibility of the invention but are not meant to limit the invention to the uses and the embodiments presented in the examples.

### Example 1

Immunizations in Conventional Mice with Classical Peptide-Carder Protein Conjugates for the Production of PrP Specific Antibodies

This example is included to show how to produce antibodies against bovine PrP in conventional mice by immunization with synthetic PrP peptides conjugated to a classical carrier protein (ovalbumin). The results show that the success rate of obtaining such antibodies is surprisingly high, taking the high similarity between mouse and bovine PrP sequences into account.

Peptides were synthesized as carboxamides by the classical Fmoc/in situ TBTU/HOBt activation solid phase method on Rink-MBHA resins as described in example 4. An additional cysteine residue was coupled at the N-terminal amino acid. After synthesis, work-up and analysis by HPLC-MS, the peptide was coupled to maleimide-derivatised ovalbumin by the following method:

Ovalbumin (Sigma, grade V, A-5503) was dissolved at 10 mg/ml in 0.1 M sodium hydrogencarbonate, pH 8.2 and mixed with 0.4 ml BMPS (N-(β-maleimidopropyloxy) succinimide ester, Pierce 22298)(5 mg/ml in N-methylpyrrolidone) pr. ml ovalbumin solution. This was incubated for 1 hour at room temperature and then desalted on a PD10 column (Amersham Biosciences) in 0.05 M sodium acetate following the manufacturer's instructions. The resulting maleimide-coupled ovalbumin was then mixed directly with 1-5 mg freeze-dried peptide pr. 5 mg ovalbumin, the exact amount of peptide depending on the reactivity of the peptide.

After 2 hours of coupling at room temperature with end-to-end mixing, the resulting complex was analysed by SDS-PAGE. A succesful coupling is seen here as a shift towards higher molecular weights of the ovalbumin bands (see FIG. 2). Immunizations were performed by subcutaneous injection of 100 μg conjugate in 200 μl 1:1 suspension of peptide in Freund's incomplete adjuvant, using 4 animals (Balb/C mice) for each peptide-conjugate. Injections were performed in the skin at the back of the neck, and each mouse received three injections with 14 days interval. Hereafter, mice were tail-bled 10 days after the last injection and the serum was analysed by ELISA (using the free (non-conjugated) immunization peptide as antigen), to give an estimate of the peptide titre attained, and then analysed against reboPrP by Western blotting with the 6H4 monoclonal mouse antibody from Prionics AG as the positive control (used 1/5000) and a pre-immune mouse serum as the negative control. All mouse sera were tested at 1/200. Blots were visualised by incubating with alkaline phosphatase coupled goat anti mouse immunoglobulins (DAKO) 1/1 000 for one hour followed by staining with NBT/BCIP tablets from Roche. The following PrP peptides were used (numbers indicate mean end-point titres obtained for each mouse in each group):

| Peptide/Mouse no. | 1 | 2 | 3 | 4 |
|---|---|---|---|---|
| PrP peptide 1 | 40.500 | 40.500 | 40.500 | 40.500 |
| PrP peptide 2 | 121.500 | 40.500 | 13.500 | 13.500 |
| PrP peptide 3 | 13.5000 | 13.500 | 13.500 | 40.500 |
| PrP peptide 4 | 4.500 | 13.500 | 13.500 | 40.500 |
| PrP peptide 5 | 121.500 | 40.500 | 40.500 | 40.500 |
| PrP peptide 6 | 13.500 | 500 | 13.500 | 1.500 |
| PrP peptide 7 | 121.500 | 13.4500 | 40.500 | 40.500 |

where
PrP peptide 1 is CGQGGTHGQWNKP
PrP peptide 2 is CGQWNKPSKPKTN
PrP peptide 3 is CGGLGGYMLGSAMSRPLIH
PrP peptide 4 is CGGRESQAYYQRGAS
PrP peptide 5 is CGSDYEDRY
PrP peptide 6 is CYYRPVDQYS and
PrP peptide 7 is CTQYQRESQAYYQRG

As seen here, titres reached the 10⁵ range and were typically above 10⁴ which is very satisfactory after three immunisations. These results were furthermore repeated for peptide 1, 4 and 6 in NMRI mice obtaining the same or even higher titres.

A representative example of an immunization result as analysed by Western blotting is depicted in FIG. 3, which in addition to some of above-mentioned peptides shows results of immunizations with 6H4 peptide (CDYEDRYYRE) and X-peptide (CWGQGGTHGQWNKPSK)

It was a surprising finding of these experiments that the peptide-ovalbumin conjugates led to the production of high-titered, PrP-reactive antibodies in all groups except the peptide 5 group.

### Example 2

Coupling Peptides to Backbones

Solely to illustrate the usefulness of a dipyridyldisulfide coupling method for coupling peptides to a backbone, model experiments were performed using peptides for which antibodies were already available.

The basis of this method is the generation, either in the backbone peptide or in the antigenic peptide of an activated thiol group either by activating a free thiol (from cysteine) by the Aidrithiol reagent (2,2-dipyridyldisulfide). The PrP peptide acetyl-CWGQGGTHGQWNKPSK was coupled to the backbone CVAKLEAKVACLEAKVAKLEAKG (with and without N-terminal palmitate) and analysed by Western blotting using a previously produced antibody raised against the ovalbumin-coupled peptide. First, the PrP peptide was reacted with 2,2-dipyridyldisulfide in solution, desalted on PD10 (Amersham Biosciences) and then reacted with the backbone in solution. The extent of coupling could be followed by measuring the release of pyridine-2-thione ar 343 nm: First, 10 mg PrP peptide was dissolved in 2 ml 0.1 M sodium phosphate, pH 8 and mixed with 2 ml Aldrithiol (5 mg/ml in N-methylpyrrolidone) and incubated for 30 minutes at room temperature. After this a sample was analysed by HPLC where pyridylsulfide-derivatised peptide showed up as a peak eluting later than the peptide. To drive the reaction to completion, an additional 25 mg Aldrithiol was added and incubation was performed as before. This was followed by PD10 (Amersham Biosciences) desalting in water and mixing with the backbone peptide. Backbone peptide was dissolved to 2 mg/ml in water and 5.4 mg activated, freeze-dried PrP-peptide was added to 500 ul backbone peptide solution adding also 100 μl acetonitril and 100 μl 0.1 M sodium acetatete, pH 5 to aid solution. This was incubated for 2 hours at room temperature. Determination of the absorption at 343 nm of the reaction mixture revealed that the reaction was finished after 10 minutes of incubation with a yield of around 30% which did not increase after up to 2 hours of incubation. The resulting conjugate was analysed by western blotting, using an antibody prepared previously against the PrP peptide (see FIG. 4).

A similar reactivity can be achieved introducing Npys-protected cysteine in either of the sequences. Fmoc-Cys(Npys) can be obtained from Novabiochem (Switzerland). In both cases, it should be remembered that the resulting conjugate consists of antigenic peptide linked to the backbone through a reduction-sensitive disulfide bond. As can be seen from FIG. 4 conjugates are obtained that contain the antigenic peptide of interest in the expected molecular weight range. As can also be seen these conjugated have a very high tendency to aggregate; this is considered an advantage with respect to achieving high multimericity and with respect to achieving a high degree of structural stabilisation.

### Example 3

The Design of Preferred Composite Peptides According to the Invention

Solely to illustrate the principles underlying the design of the composite peptides of the invention some core structures are described with reference to FIGS. 5, 6 and 7.

One favoured model for a major conformational transition taking place in the PrP^{C} to PrP^{Sc} transformation is depicted in FIG. 5A/B and is based on the conformational epitope corresponding to the PrP^{Sc}-specific antibody 15B3 (Korth, C., et al., 1997, Nature 390, 74-77): The most N-terminal α-helix of the globular domain of PrP^{C} is changed into two β-strands making up a new, PrP^{Sc}-specific 4-strand β-strand (FIG. 5B), in which strands 2 and 3 derive from the α-helix. This means that a simple, PrP^{Sc}-specific epitope mimic can be formed by linking the sequence of β-strand 2 with that of β-strand 3 in a conformation resembling the hairpin antiparrallel structure found in PrP^{Sc} (FIG. 5C). To achieve a coupling of this epitope mimic to a backbone, the structure was equipped with an N-terminal cysteine residue. When coupled side-by-side on a backbone peptide the structure is further stabilised.

Another way to design a structure that mimics the PrP^{Sc}-specific 15B3-epitope is depicted in FIG. 6, where two such epitopes are delineated, the first one being composed of the two 15B3 epitope segments corresponding to the turn at the “top” of the four-strand β-sheet (the first 15B3 epitope, 15B3/I) plus the segment terminating β-strand 4 (15B3/2); as can be seen this structure comprises the two different peptides strands running in opposite directions (above B) coupled together by a maleimide (BMPS)-thiol (cysteine) bond and carrying a fatty acid (the dotted line) at the C-terminal end of the 15B3/I peptide. In this way the fatty acid containing peptide acts as a backbone for the other peptide at the same time as being part of the epitope mimic, and by positioning the fatty acid at the extreme end of this peptide the correct non-covalent assembly of the whole construct is presumed to occur with the fatty acid being inside the assembly and the peptide parts exposed at the surface. In FIG. 6C, β-strand 1 is coupled to the first 15B3 epitope (15B3/I) in opposite directions, again mimicking the situation on the 15B3 epitope, and again, the fatty acid is used to ensure the right orientation of the construct in the aggregates being formed. Finally, FIG. 7 shows some possibilities of combining 2 (B and C) and 3 (A) peptides on a backbone, showing the possibility of different directions and arrangements. Again BMPS may be used for N-oriented couplings while C-terminal coupling is done by sequential synthesis or, more preferred by active ester activation of the side-chain protected peptide.

In these structures the peptide representing 15B3/1 is always present as this is the PrP^{Sc}-defining part of the epitope, resulting in PrP^{Sc}-specific composite peptides when combined with any other parts of the 15B3-epitope or both parts at the same time.

### Example 4

Peptide Constructs for Immunization I: A Conjugate According to the Invention Containing One PrP^{Sc}-Peptide

As an example of this kind of structure, a conjugate comprising the bovine prion peptide WGQGGTHGQWNKPSK (bo101-115) coupled to the following backbone lipopeptide KRGGKRGGK-(palm) was synthesized and used. The BSE-peptide is coupled to the side chains of the two lysine residues that have free epsilon-amino groups, forming a conjugate containing two copies of one BSE-peptide. This peptide derives from the conformationally labile N-terminal part of the globular part of PrP and is envisaged to be stabilised in a PrP^{Sc}-like conformation by its side-by-side positioning on the backbone peptide. The region in PrP comprising this peptide is furthermore the region being processed differently in different strains and species of PrP and is therefore not present in certain types of PrP^{Sc} while present in others. The example is not limited to this bovine peptide but may also be applied to the corresponding human (hu89-103: WGQGGTHSQWNKPSK) and ovine (ov93-107: WGQGGSHSQWNKPSK) peptides and may furthermore be extended to the use of the following PrP peptides being from a conformationally labile region close to that of the above peptides and with a proven species specificity derived from the subtle sequence differences between species:

bo114-123: SKPKTNMKHV

hu103-112: SKPKTNMKHM

ov106-115: SKPKTNMKHV

and peptides being part of these sequences or polymorphs thereof.

Also very useful for inclusion into composite peptides of this type are various structure-supported PrP^{Sc}-related peptides, including

bo143-162: SAMSRPLIHFG-dib-SDYEDRYYR (see FIG. 5)

bo114-123: dib-GGTHGQW NKPSKPKTNM KHV

bo153-171: dib-GSDYEDRY YRENMHRYPN Q

bo139-176: YM LGSAMSRPLI HFG-dib-SDYEDRY YRENMHRYPN QVYYRP

in which “dib” represents a 4(2-aminoethyl) 6-dibenzofuranpropionic acid residue (Neosystem Groupe SNPE) or another beta-strand supporting residue. These peptides are characterised by having a preferential conformation, aided by the structure-support element and subsequently further supported by their side-by-side coupling to the backbone peptide, said conformation being specific for the conformation of the peptide sequence in PrP^{Sc} and not found in PrP^{C}.

Synthesis of Peptides

The following is provided as an example of a particularly preferred way of synthesizing conjugates of this type.

The backbone peptide (KR(Pbf)GGKR(Pbf)GGK-(palm)) was synthesized by the Fmoc/tertButyl solid phase peptide synthesis strategy on a Rink-MBHA-resin from Novabiochem, using Mtt-side chain protected lysine and Pbf-side chain protected arginine in addition to glycine, all from Novabiochem. The synthesis was performed on a semiautomatic peptide synthesizer from Abimed GmbH using TBTU/HOBt in situ activation and 20% piperidin in NMP for Fmoc-deprotection using standard conditions (Chan, W. C., and White, P. D., 2000, Fmoc Solid Phase Peptide Synthesis, Oxford University Press). After coupling of the first K(Mtt), the Mtt group was removed by 1% TFA, 5% triisopropylsilane in NMP and the side chain amino group was then palmitoylated by acylation with palmitic acid (Merck) in the presence of TBTU and HOBt until the colour of a sample of the resin went from orange/red to white by reaction with TNBS (trinitrobenzenesulfonic acid (Sigma). Then the synthesis was resumed, initiating with a piperidine incubation (20% piperidine in NMP) to remove the Fmoc-group. After completion of the peptide, the α-amino group was acetylated by acetic anhydride followed by washing and then by removal of the two remaining Mtt-groups by TFA/triisopropylsilane as above. This deprotects the two ε-amino groups of the lysine residues but leaves the arginine side-chain protection intact. Then the free amino groups were reacted with BMPS (Pierce) in NMP until a negative TNBS-test was obtained, followed by cleavage of the whole peptide as the amide from the resin by 95% TFA/water with appropriate scavengers and work-up following standard procedures (Chan, W. C., and White, P. D., 2000, Fmoc Solid Phase Peptide Synthesis, Oxford University Press).

HPLC-MS (Shimadzu LCMS 2010, ESI-MS) was performed on a sample of the peptide and the molecular weight was confirmed.

For the subsequent coupling of the BSE-peptide, this was first synthesized with a N-terminal cysteine by standard Fmoc/tBu chemistry on solid phase, cleaved as the amide and worked up following standard procedures. The actual coupling was performed by dissolving the backbone peptide to 2 mg/ml in water and mixing 1 ml of this solution with 5 mg of the freeze-dried BSE-peptide and incubating for 2 hours at room temperature with stirring. This was then freeze-dried directly. In the resulting composite peptide, the BSE-peptide was bound to the backbone peptide through a non-reducible thioether bond. In another equally appropiate procedure, Cys(Npys) was used instead of BMPS to derivatize the free ε-amino groups of the lysines, and the resulting backbone peptide was reacted with the BSE-peptide as above. This gave rise to a composite peptide in which the BSE-peptide was bound to the backbone peptide through a reducible disulfide bond. HPLC-MS (Shimadzu LCMS 2010, ESI-MS) was performed on a sample of both peptide compounds and the molecular weight was confirmed.

Immunization Procedures

Mice, such as female 6- to 8-weeks old BALB/c mice or (CF1xBALB/c)F1 mice, were immunized 3 times with 14 days intervals with different BSE peptide constructs mixed 1+1 with Freunds adjuvant. The mice were immunized subcutaneously with 16 μg of the peptide. The mice were bled before the first immunization and 12 days after each immunization. Sera were collected from the bleedings and tested in ELISA for antibody reactivity against BSE peptides. Likewise rabbits were immunized with app. 50 μg of BSE peptide constructs.

Antibody Reactivities in ELISA Assays

Peptides (0.25 μg/ml) were coated to Maxisorp microtiter plates (Nunc, Roskilde, Denmark) in 100 mM NaHCO₃ at pH 9.6. All coatings were performed overnight at 4° C. To avoid background reactivity with the backbone part of the composite peptides used for immunizations, coating was performed with either the free peptides or peptides coupled to a conventional carrier protein, typically ovalbumin, by maleimide-cysteine chemistry. The wells were washed four times in washing buffer (0.5 M NaCl, 3 mM KCl, 1 mM KH₂PO₄, 8 mM Na₂HPO₄, and 1% Triton X-100). This washing procedure was done after each of the following incubation steps: 1) serum samples, 1% (v/v) in incubation buffer (washing buffer plus 15 mM bovine albumin, pH 7.2), were incubated for 1 hr at room temperature; 2) 100 μl per well of horseradish peroxidase-conjugated rabbit anti-murine IgG antibodies (DAKO, Copenhagen, Denmark) diluted in incubation buffer, were added at room temperature for 1 hr. Enzyme activities were quantitated after the addition of 100 μl per well of 1,2-phenyldiamine hydrochloride (0.67 mg/ml) (DAKO) dissolved in 100 mM citric acid-phosphate buffer, pH 5.0, containing 0.015% (v/v) H₂O₂. The reactions stopped after 30 min by adding 50 μl per well of 2.5 M H₂SO₄, and the optical densities were measured in an enzyme-linked immunosorbent assay (ELISA) scanner at 492 nm. All tests were performed in duplicate.

Serial dilution series were carried out with positive sera in order to determine the titre of positive sera. Some seropositive mice were selected for hybridoma production by standard procedures.

Validation of Positive Antibodies

Seropositive sera and/or monoclonal antibodies were tested by standard procedures against PrP^{Sc}-infected brain material by ELISA, filter-based assays, Western blotting or fluorescence assays, using brain material from confirmed BSE-positive cows, from scrapie-positive sheep and from CJD-diagnosed humans, depending on the peptide in question.

Positive sera or monoclonal antibodies were selected for development of a blood based PrP^{Sc}-test. This may or may not comprise an initial extraction step to extract PrP^{Sc} in sufficient quantities to allow its quantitation.

Blood Based BSE Test (Sandwich Like Type)

Seropositive sera or monoclonal antibodies (10 μg/ml) were coated to Maxisorp microtiter plates (Nunc, Roskilde, Denmark) in 100 mM NaHCO₃ at pH 9.6. All coatings were performed overnight at 40° C. The wells were washed four times in washing buffer (0.5 M NaCl, 3 mM KCl, 1 mM KH₂PO₄, 8 mM Na₂HPO₄, and 1% Triton X-100). This washing procedure was done after each of the following incubation steps: 1) test serum samples, 1% (v/v) in incubation buffer (washing buffer plus 15 mM bovine albumin, pH 7.2), were incubated for 1 hr at room temperature; 2) 100 μl per well of biotinylated seropositive sera or monoclonal antibodies diluted in incubation buffer, were added at room temperature for 1 hr. 3) 100 μl per well of horseradish peroxidase-conjugated streptavidin (DAKO, Copenhagen, Denmark) diluted in incubation buffer, were added at room temperature for 1 hr. Enzyme activities were quantitated after the addition of 100 μl per well of 1,2-phenyldiamine hydrochloride (0.67 mg/ml) (DAKO) dissolved in 100 mM citric acid-phosphate buffer, pH 5.0, containing 0.015% (v/v) H₂O₂. The reactions stopped after 30 min by adding 50 μl per well of 2.5 M H₂SO₄, and the optical densities were measured in an enzyme-linked immunosorbent assay (ELISA) scanner at 492 nm. All tests were performed in triplicate. Various procedures were applied to amplify the signal including standard biotin-streptavidin amplification systems. Dilutions of infected material (standard) were tested on the same plate.

Blood Based BSE Test (Competition Like Type)

Synthetic peptide constructs (0.25 μg/ml) were coated to Maxisorp microtiter plates (Nunc, Roskilde, Denmark) in 100 mM NaHCO₃ at pH 9.6. All coatings were performed overnight at 40° C. The wells were washed four times in washing buffer (0.5 M NaCl, 3 mM KCl, 1 mM KH₂PO₄, 8 mM Na₂HPO₄, and 1% Triton X-100). This washing procedure was done after each of the following incubation steps: 1) test whole blood, serum or plasma samples, 1-50% (v/v) in incubation buffer (washing buffer plus 15 mM bovine albumin, pH 7.2) mixed with diluted biotinylated seropositive sera or monoclonal antibody, were incubated for 2 hr at 37° C.; 2) 100 μl per well of horseradish peroxidase-conjugated streptavidin (DAKO, Copenhagen, Denmark) diluted in incubation buffer, were added at room temperature for 1 hr. Enzyme activities were quantitated after the addition of 100 μl per well of 1,2-phenyldiamine hydrochloride (0.67 mg/ml) (DAKO) dissolved in 100 mM. citric acid-phosphate buffer, pH 5.0, containing 0.015% (v/v) H₂O₂. The reactions stopped after 30 min by adding 50 μl per well of 2.5 M H₂SO₄, and the optical densities were measured in an enzyme-linked immunosorbent assay (ELISA) scanner at 492 nm. All tests were performed in triplicate. Seropositive sera or monoclonal antibody mixed with buffer served as a control.

Percent inhibition of seropositive sera or monoclonal antibody binding was calculated from the formula: 100−((ELISA ODₛₐₘₚₗₑ/ELISA OD_{control})×100). Significant inhibition reflects a BSE positive sample. Standard curves were obtained with infected material in serial dilutions.

Blood Based BSE Test (Fluorescence-Based)

An adequate purified polyclonal antibody or an appropriate monoclonal antibody, prepared against PrP-relevant peptides as detailed above is used in a homogenous assay where the blood sample is brought into contact with the antibody together with a fluorescently labelled derivative of the peptide antigen in question. After a short incubation period the polarization ability of the peptide-coupled flourophor is then measured in fluorescence polarization reader. As this parameter depends on the rotational freedom of the flourescent molecule, the signal from the flourescent peptide will vary depending if the peptide is bound by the antibody or if it is competed off into free solution by a specifically binding PrP^{Sc} present in the blood sample. Thus, with an adequate PrP^{Sc}-standard, the fluorescence polarisation value expresses how much PrP^{Sc} is present in the sample.

### Example 5

Peptide Constructs for Immunization II: Conjugate Containing Two Different PrP^{Sc}-Peptides Using One of the Peptides as the Backbone

As an example, three peptides representing different parts of the conformational 15B3 PrP^{Sc}-specific epitope reported in the literature (Korth et al., 1997, Nature 390, 74-77) are combined to provide composite peptides representing PrP^{Sc}-specific spatial structure. These peptides derive from the region making up the four-strand β-sheet postulated to be specific for PrP^{Sc} and constituting the part of the 15B3-epitope being different in PrP^{C} and PrP^{Sc}. It should be noted that care has been taken to ensure the right orientation of the two peptides making up the composite peptide in order to resemble exactly the arrangement of the corresponding peptides in the PrP^{Sc}-structure (see FIG. 6 and example 3).

It is to be understood that while the example deals with boPrP-derived peptides and hence with detection of BSE-derived PrP^{Sc}, the example is likewise applicable to human and ovine PrP^{Sc}-detection, using the equivalent sequences from these two proteins. One composite peptide comprises bmps-GSDYEDRYYK-(palm) (bo153-161 with an additional C-terminal lysine to which palmitate is coupled and a BMPS-labeled N-terminal) as the backbone peptide onto which CYYRPVDQYSN (bo173-182 with an additional N-terminal cysteine) is coupled. As can be seen from FIG. 6 this mimics the arrangement of the peptide turn C-terminally to the second β-strand of the PrP^{Sc}-specific β-sheet and the peptide stretch C-terminally to the fourth β-strand of the PrP^{Sc}-sheet, with opposite directions of the peptide chains. Furthermore, by placing palmitate on the C-terminal amino acid of the second β-strand related peptide, the immunogen will orient itself with the β-strand four-related peptide as the outermost part of the immunogen. For this particular construct a similar composite peptide with palmitate introduced at the N-terminus of one of the two peptides or at the C-terminus of the β-strand four-related peptide will constitute other equally preferred composite peptides.

Another composite peptide contains bmpsYMLGSAMSRPK-(palm) (bo139-148 with an additional C-terminal lysine to which is coupled palmitate and a BMPS-labele N-terminal) as the backbone peptide onto which CGSDYEDRYYRE (bo153-163 with an additional N-terminal cysteins) is coupled. As can be seen from FIG. 6 this mimics the spatial arrangement of the first P-strand and part of β-strand 2, represented with the first peptide and the peptide turn C-terminally to the second γ-strand of the PrP^{Sc}-specific β-sheet (the second peptide of the construct), again with opposite directions of the peptide chains. Here, the palmitate ensures the inwards orientation of the first peptide in the immunogen, resembling the situation in the four-strand β-sheet of the PrP^{Sc}-structure. This is the preferred position of the palmitate in this construct.

Synthesis of Peptides

This was done by the same methods as described above, using Mtt-protected C-terminal lysine for the introduction of palmitate (deprotecting and acylating the ε-aminogroup before continuing synthesis). Molecular weights were confirmed by HPLC-MS (Shimadzu LC-MS 2010).

Immunizations, validations and immunoassays are then performed as described above and useful antibodies/immunoassays are expected to be obtained.

### Example 6

Peptide Constructs for Immunization III: Conjugates Containing Two or More Different BSE-Peptides

As an example illustrating how the peptides making up the PrP^{Sc}-specific 15B3-epitope can be combined into a composite peptide as described herein the following compounds were produced:

(the three peptide sequences making up the composite 15B3 epitope (Korth et al., 1997, Nature 390, 74-77) being 15B3/1: bo153-159, GSDYEDR, 15B3/2: bo173-181, YYRPVDQYS, 15B3/3: bo226-237, ITQYQRESQAYY, respectively).

As in the other examples, the equivalent peptides from human and ovine PrP can be used to develop immunogens specific for these species.

palm-VAKLEAKVACLEAKVACKG K G

in which 15B3/1 is coupled to K and 15B3/2 and 15B3/3 are coupled to either of the two cysteines. As can be seen in FIG. 7 this leads to structures in which the three 15B3-peptides are arranged spatially in the same manner as in the PrP^{Sc}-specific 15B3-epitope, also retaining the different orientations of the peptide stretches in this epitope.

Furthermore, the backbone peptide of this example is stabilised conformationally being an amphipathic α-helix, lending further structural stabilization to the attached PrP-peptides.

palm-VAKLEAKVACLEAKVAKLEAKVACKG KG

in which 15B3/1 is coupled to K and 15B3/2 or 15B3/3 is coupled to both cysteines. As can be seen in FIG. 7 the resulting structures combine 15B3/1 and 15B/2 or, in a separate composite peptide 15B3/1 and 15B3/3 in a manner resembling their arrangement and orientations in the PrP^{Sc}-specific 15B3-epitope, each composite peptide representing part of the structure (a part that in each case contains the PrP^{Sc}-specific features) and each composite peptide presenting two copies of the PrP^{Sc}-specific structure. Furthermore, the backbone peptide of this example is stabilised conformationally being an amphipathic α-helix, lending further structural stabilization to the attached PrP-peptides.

The synthesis is performed essentially as described above, starting with the backbone peptide, which is synthesized with the N-terminal palmitate before Mtt is removed when the peptide is still attached to the solid phase resin; hereafter the 15B3/1-peptide is synthesized by solid-phase synthesis on the liberated ε-amino groups. Then the whole peptide (now branched) is cleaved from the solid phase, in the process deprotecting the thiol groups of the cysteines (should be trityl protected). Hereonto are then coupled the N-terminally BMPS-derivatised 15B3/2 and 15B3/3 peptides as described above.

With the first peptide, a mixture of composite peptides will result, arising from different combinations of the two 15B3-peptides (2 and 3) with the already attached 15B3/1-peptide (1): 2,3,1; 3,2,1; 2,2,1; 3,3,1. All of these peptides, as well as the mixture itself are thought to be useful immunogens for the production of PrP^{Sc}-specific antibodies as outlined above.

As another example illustrating an important composite peptide, the K-positions are used for the coupling of a peptide selected from those described above in the paragraph “Conjugates containing one BSE-peptide” in combination with 15B3-peptides. Specifically SAMSRPLIHFG-dib-SDYEDRYYR is synthesized on the K of the backbone peptides above and combined with any or both of 15B3/1 and 15B3/2.

Immunizations, validations and immunoassays were then performed as described above and useful antibodies/immunoassays are expected to be obtained.

### Example 7

A Composite Peptide Construct for the Direct Detection of PrP^{Sc}

A composite peptide compound as the one described in Example 4 above will be synthesized. As the prion peptides of this compound contain two tryptophan residues each they will exhibit an intrinsic fluoresence that will report on the relative positions of the tryptophan residues in the individual peptide chains as well as between chains.

For other prion peptides, tryptophan or tryptophan-quench pairs (one quencher building block being abbreviated aedans (=5-((2-(tBoc)-glutamylaminoethyl)amino)naphthalene-1-sulfonic acid) from Molecular Probes) will be inserted into the prion peptides at strategic points that will reflect the conformation of the peptides.

An assay based on such compounds will be developed, using PrP^{Sc} spiked and un-spiked samples as positive and negative samples, respectively, and varying the following parameters to obtain the most sensitive peptide compound:

type of prion peptide

type and position of fluorescence-quench pairs

insertion of conformation-supporting or relaxing residues

solvent conditions for sample and/or the peptide compound

treatment of both components and the mixture by sonication, ultrasound, heat or other means.

The assay will be analysed by fluorescence spectroscopy correcting for the background being caused by the presence of PrP^{C} in the samples.

1. A conjugate comprising two or more peptides or peptide fragments optionally linked to a backbone and the peptides or peptide fragments are spatially positioned relative to each other so that they together form a non-linear sequence which mimics the tertiary structure of one or more PrPSc-specific epitopes as evidenced by the test described herein.

2. A conjugate comprising two or more peptides or peptide fragments optionally linked to a backbone and the peptides or peptide fragments are spatially positioned relative to each other so that they together form a non-linear sequence which mimics the tertiary structure of PrPSc and has the same or a higher degree of conformational sensitivity to PrPSc as one or more conformationally sensitive regions of prpc as evidenced by the test described herein.

3. A conjugate according to claim 1, wherein the peptides or peptide fragments comprise from 2 to 150 amino acids.

4-5. (canceled)

6. A conjugate according to claim 1, wherein at least one of the two or more peptides or peptide fragments is a prion peptide or a prion peptide fragment.

7-8. (canceled)

9. A conjugate according to claim 6, wherein the prion peptide or prion peptide fragment has a primary structure corresponding to a bovine PrP SEQ. ID No.1, a ovine PrP SEQ. ID No. 2, a human PrP SEQ. ID No. 3, or polymorphs or fragments thereof.

10. A conjugate according to claim 1, wherein the peptides or peptide fragments are selected from the group consisting of SEQ. ID No. 4, SEQ. ID No. 5, SEQ. ID No. 6, SEQ. ID No. 7, SEQ. ID No.8, SEQ. ID No.9, SEQ. ID No. 10, SEQ. ID No.11, SEQ. ID No. 12 and SEQ. ID No. 13.

11. A conjugate according to claim 1, wherein the peptides or peptide fragments are selected from the group consisting of SEQ. ID No. 14, SEQ. ID No. 15 and SEQ. ID No. 16.

12. A conjugate according to claim 1, wherein the peptides or peptide fragments are selected from the group consisting of SEQ. ID No. 17, SEQ. ID No. 18, SEQ. ID No. 19 and SEQ. ID No. 20.

13. A conjugate according to claim 1, wherein the peptides or peptide fragments are selected from the group consisting of SEQ. ID No. 21, SEQ. ID No. 22, SEQ. ID No. 23, SEQ. ID No. 24, SEQ. ID No. 25 and SEQ. ID No. 25 A-F.

14. A conjugate according to claim 1, wherein the peptides or peptide fragments have a sequence corresponding to SEQ. ID No. 26.

15. A conjugate according to claim 1, wherein the peptides or peptide fragments are selected from the group consisting of SEQ. ID No. 27, SEQ. ID No.28, SEQ. ID No.29, SEQ. ID No.30, SEQ. ID No.31, SEQ. ID No. 32, SEQ. ID No.33, SEQ. ID No.34, SEQ. ID No.35, SEQ. ID No.36, SEQ. ID No.37, SEQ. ID No.38, SEQ. ID No. 39, SEQ. ID No. 40, SEQ. ID No. 41, SEQ. ID No. 42, SEQ. ID No. 43 and SEQ. ID No. 44.

16. A conjugate according to claim 1, wherein a β-strand inducing building block is introduced in the amino acid sequence of the peptides or peptide fragments.

17. A conjugate according to claim 1, wherein at least two of the two or more peptides or peptide fragments have identical amino acid sequences.

18. A conjugate according to claim 1 wherein at least one of the two or more peptides or peptide fragments is a T-cell helper epitope.

19. A conjugate according to claim 1 which has the formula:
(F)ₘ(X)ₚ
wherein F is independently the same or different prion peptide or prion peptide fragment;
X is the same or different amino acid residue or peptide;
m is an integer from 2 to 10 inclusive;
and p is an integer from 0 to 10 inclusive;
such that X and F together form a conjugate, provided that the resulting conjugate is not prion peptide or a prion peptide fragment.

20-28. (canceled)

29. A conjugate according to claim , wherein a backbone is contained in the conjugate.

30. A conjugate according to claim 29, wherein the backbone is a non-dendritic peptide backbone.

31. A conjugate according to claim 29, wherein the backbone is a lipopeptide.

32. A conjugate according to claim 31 wherein the backbone has the structure
(LiP)ₓ(A)ₙ
wherein
A is an amino acid which may be the same or different and may contain one or more attachment points;
n is an integer from 2 to 150 such that (A)ₙ is a chain of amino acids which 5 may be branched or linear;
Lip is a lipophilic moiety which is linked to A through a bond or a linker; and x is an integer from 1 to 10 such that 1-10, same or different, Lip could be joined to the backbone.

33-49. (canceled)

50. A conjugate according to claim 29, wherein the backbone has a structure selected from the group consisting of:
a) palm-KVAKLEAKVAKLEAKVAKLEAKG
b) palm-VACLEAKVACLEAKVACLEAKGKGKG
c) palm-VAKLEAKVACLEAKVACKGKG
d) palm-VAKLEAKVACLEAKVAKLEAKVAC
e) KGGKRGGK-(palm)
f) palm-VAKLEAKVACLEAKVACKG K G
g) palm-VAKLEAKVACLEAKVAKLEAKVACKG KG
h) palm-PrP
i)palm-PrP fragment
j) GSDYEDRYYK-(palm)
k) YMLGSAMSRPK-(palm)
with the peptide side chains at one or more positions being optionally protected by protecting groups.

51-55. (canceled)

56. A conjugate according to claim 1 further comprising a marker.

57-64. (canceled)

65. A method for the production of antibodies against PrP^{Sc}, the method comprising immunizing an animal with a conjugate according to claim 1.

66-68. (canceled)

69. An antibody against PrP^{Sc} obtainable by the method claimed in any of claim 65.

70-71. (canceled)

72. A method for detection of PrP^{Sc} in a sample comprising i) optionally, treating the sample with Proteinase K ii) contacting the sample with an antibody according to claim 69 iii) detecting any PrP^{Sc} which is bound to the antibody.

73-78. (canceled)

79. A method for identifying PrP^{Sc} by means of a substance which undergoes conformational change when contacted with PrP^{Sc}, the method comprising i) incubation of the substance with PrP^{Sc} in a structure-relaxing solvent, ii) measuring any conformational change of the substance by conformation-specific antibodies or by detection of changes in the fluorescence of an environmentally sensitive fluorophore coupled to the substance.

80. (canceled)

81. A pharmaceutical composition comprising a conjugate according to claim 1.

82. A pharmaceutical composition comprising an antibody according to claim 69.

83. A vaccine composition comprising a conjugate according to claim 1.

84. A vaccine composition comprising an antibody according to claim 69.

85. A method for treating and/or preventing Creutzfeldt-Jakobs disease, kuru, Gerstmann-Straussler-Sheinker disease, fatal familial insomnia and transmissible spongiform encephalopathies, such as bovine spongiform encephalopathy in cattle, scrapie in sheep, chronic wasting disease in deer and elk and transmissible encephalopathies in mink, cat and other animals, the method comprising administering to an animal an effective amount of a conjugate according to claim 1.

86. A method for treating and/or preventing Creutzfeldt-Jakobs disease, kuru, Gerstmann-Straussler-Sheinker disease, fatal familial insomnia and transmissible spongiform encephalopathies, such as bovine spongiform encephalopathy in cattle, scrapie in sheep, chronic wasting disease in deer and elk and transmissible encephalopathies in mink, cat and other animals, the method comprising administering to an animal an effective amount of an antibody according to claim 69.
